SEQUENCE LISTING (15 sequences)

SEQ ID NO: 1 - DNA, 789 bp, Nostoc sp. PCC73102
  CDS (1)..(789)
ttgaatttttgtgataaaccagttagctattatgttgcaatagagcaattaagtgctaaagaagatactgtttgggggctggtgattgtcatagtaattattagtctttgggtagctagtttggcttttttactagctattaattatgccaaagtcccaatttggttgatacctattgcaatagtttggcaaatgttcctttatacagggctatttattactgcacatgatgctatgcatgggtcagtttatcgtaaaaatcccaaaattaataattttatcggttcactagctgtagcgctttacgctgtgtttccatatcaacagatgttaaagaatcattgcttacatcatcgtcatcctgctagcgaagttgacccagattttcatgatggtaagagaacaaacgctattttctggtatctccatttcatgatagaatactccagttggcaacagttaatagtactaactatcctatttaatttagctaaatacgttttgcacatccatcaaataaatctcatcttattttggagtattcctccaattttaagttccattcaactgttttatttcggaacatttttgcctcatcgagaacccaagaaaggatatgtttatccccattgcagccaaacaataaaattgccaacttttttgtcatttatcgcttgctaccactttggttatcatgaagaacatcatgagtatccccatgtaccttggtggcaacttccatctgtatataagcagagagtattcaacaattcagtaaccaattcgtaa

SEQ ID NO: 2 - protein, 262 aa, Nostoc sp. PCC73102
LNFCDKPVSYYVAIEQLSAKEDTVWGLVIVIVIISLWVASLAFLLAINYAKVPIWLIPIAIVWQMFLYTGLFITAHDAMHGSVYRKNPKINNFIGSLAVALYAVFPYQQMLKNHCLHHRHPASEVDPDFHDGKRTNAIFWYLHFMIEYSSWQQLIVLTILFNLAKYVLHIHQINLILFWSIPPILSSIQLFYFGTFLPHREPKKGYVYPHCSQTIKLPTFLSFIACYHFGYHEEHHEYPHVPWWQLPSVYKQRVFNNSVTNS

SEQ ID NO: 3 - DNA, 762 bp, Nostoc sp. PCC73102
  CDS (1)..(762)
gtgatccagttagaacaaccactcagtcatcaagcaaaactgactccagtactgagaagtaaatctcagtttaaggggcttttcattgctattgtcattgttagcgcatgggtcattagcctgagtttattactttcccttgacatctcaaagctaaaattttggatgttattgcctgttatactatggcaaacatttttatatacgggattatttattacatctcatgatgccatgcatggcgtagtatttccccaaaacaccaagattaatcatttgattggaacattgaccctatccctttatggtcttttaccatatcaaaaactattgaaaaaacattggttacaccaccacaatccagcaagctcaatagacccggattttcacaatggtaaacaccaaagtttctttgcttggtattttcattttatgaaaggttactggagttgggggcaaataattgcgttgactattatttataactttgctaaatacatactccatatcccaagtgataatctaacttacttttgggtgctaccctcgcttttaagttcattacaattattctattttggtacttttttaccccatagtgaaccaatagggggttatgttcagcctcattgtgcccaaacaattagccgtcctatttggtggtcatttatcacgtgctatcattttggctaccacgaggaacatcacgaatatcctcatatttcttggtggcagttaccagaaatttacaaagcaaaatag

SEQ ID NO: 4 - protein, 253 aa, Nostoc sp. PCC73102
VIQLEQPLSHQAKLTPVLRSKSQFKGLFIAIVIVSAWVISLSLLLSLDISKLKFWMLLPVILWQTFLYTGLFITSHDAMHGVVFPQNTKINHLIGTLTLSLYGLLPYQKLLKKHWLHHHNPASSIDPDFHNGKHQSFFAWYFHFMKGYWSWGQIIALTIIYNFAKYILHIPSDNLTYFWVLPSLLSSLQLFYFGTFLPHSEPIGGYVQPHCAQTISRPIWWSFITCYHFGYHEEHHEYPHISWWQLPEIYKAK

SEQ ID NO: 5 - DNA, 1608 bp, Haematococcus pluvialis
  CDS (3)..(971)
ctacatttcacaagcccgtgagcggtgcaagcgctctgccccacatcggcccacctcctcatctccatcggtcatttgctgctaccacgatgctgtcgaagctgcagtcaatcagcgtcaaggcccgccgcgttgaactagcccgcgacatcacgcggcccaaagtctgcctgcatgctcagcggtgctcgttagttcggctgcgagtggcagcaccacagacagaggaggcgctgggaaccgtgcaggctgccggcgcgggcgatgagcacagcgccgatgtagcactccagcagcttgaccgggctatcgcagagcgtcgtgcccggcgcaaacgggagcagctgtcataccaggctgccgccattgcagcatcaattggcgtgtcaggcattgccatcttcgccacctacctgagatttgccatgcacatgaccgtgggcggcgcagtgccatggggtgaagtggctggcactctcctcttggtggttggtggcgcgctcggcatggagatgtatgcccgctatgcacacaaagccatctggcatgagtcgcctctgggctggctgctgcacaagagccaccacacacctcgcactggaccctttgaagccaacgacttgtttgcaatcatcaatggactgcccgccatgctcctgtgtacctttggcttctggctgcccaacgtcctgggggcggcctgctttggagcggggctgggcatcacgctatacggcatggcatatatgtttgtacacgatggcctggtgcacaggcgctttcccaccgggcccatcgctggcctgccctacatgaagcgcctgacagtggcccaccagctacaccacagcggcaagtacggtggcgcgccctggggtatgttcttgggtccacaggagctgcagcacattccaggtgcggcggaggaggtggagcgactggtcctggaactggactggtccaagcggtagggtgcggaaccaggcacgctggtttcacacctcatgcctgtgataaggtgtggctagagcgatgcgtgtgagacgggtatgtcacggtcgactggtctgatggccaatggcatcggccatgtctggtcatcacgggctggttgcctgggtgaaggtgatgcacatcatcatgtgcggttggaggggctggcacagtgtgggctgaactggagcagttgtccaggctggcgttgaatcagtgagggtttgtgattggcggttgtgaagcaatgactccgcccatattctatttgtgggagctgagatgatggcatgcttgggatgtgcatggatcatggtagtgcagcaaactatattcacctagggctgttggtaggatcaggtgaggccttgcacattgcatgatgtactcgtcatggtgtgttggtgagaggatggatgtggatggatgtgtattctcagacgtagaccttgactggaggcttgatcgagagagtgggccgtattctttgagaggggaggctcgtgccagaaatggtgagtggatgactgtgacgctgtacattgcaggcaggtgagatgcactgtctcgattgtaaaatacattcagatgcaaaaaaaaaaaaaaaaaaaaaaa

SEQ ID NO: 6 - protein, 322 aa, Haematococcus pluvialis
TFHKPVSGASALPHIGPPPHLHRSFAATTMLSKLQSISVKARRVELARDITRPKVCLHAQRCSLVRLRVAAPQTEEALGTVQAAGAGDEHSADVALQQLDRAIAERRARRKREQLSYQAAAIAASIGVSGIAIFATYLRFAMHMTVGGAVPWGEVAGTLLLVVGGALGMEMYARYAHKAIWHESPLGWLLHKSHHTPRTGPFEANDLFAIINGLPAMLLCTFGFWLPNVLGAACFGAGLGITLYGMAYMFVHDGLVHRRFPTGPIAGLPYMKRLTVAHQLHHSGKYGGAPWGMFLGPQELQHIPGAAEEVERLVLELDWSKR

SEQ ID NO: 7 - DNA, 1650 bp, Lycopersicon esculentum
  CDS (112)..(1614)
ggcacgaggaaacttttctctcttcactagctgtttacatgcttgaaatttcaagattttaggaccccatttgaagttttcttgaaacaaatattaccctgttggaaaaagatggatactttgttgaaaaccccaaataaccttgaatttctgaacccacatcatggttttgctgttaaagctagtacctttagatctgagaagcatcataattttggttctaggaagttttgtgaaactttgggtagaagtgtttgtgttaagggtagtagtagtgctcttttagagcttgtacctgagaccaaaaaggagaatcttgattttgagcttcctatgtatgacccttcaaaaggggttgttgtggatcttgctgtggttggtggtggccctgcaggacttgctgttgcacagcaagtttctgaagcaggactctctgtttgttcaattgatccgaatcctaaattgatatggcctaataactatggtgtttgggtggatgaatttgaggctatggacttgttagattgtctagatgctacctggtctggtgcagcagtgtacattgatgataatacggctaaagatcttcatagaccttatggaagggttaaccggaaacagctgaaatcgaaaatgatgcagaaatgtataatgaatggtgttaaattccaccaagccaaagttataaaggtgattcatgaggaatcgaaatccatgttgatatgcaatgatggtattactattcaggcaacggtggtgctcgatgcaactggcttctctagatctcttgttcagtatgataagccttataaccccgggtatcaagttgcttatggcattttggctgaagtggaagagcacccctttgatgtaaacaagatggttttcatggattggcgagattctcatttgaagaacaatactgatctcaaggagagaaatagtagaataccaacttttctttatgcaatgccattttcatccaacaggatatttcttgaagaaacatcactcgtagctcgtcctggcttgcgtatagatgatattcaagaacgaatggtggctcgtttaaaccatttggggataaaagtgaagagcattgaagaagatgaacattgtctaataccaatgggtggtccacttccagtattacctcagagagtcgttggaatcggtggtacagctggcatggttcatccatccaccggttatatggtggcaaggacactagctgcggctcctgttgttgccaatgccataattcaatacctcggttctgaaagaagtcattcgggtaatgaattatccacagctgtttggaaagatttgtggcctatagagaggagacgtcaaagagagttcttctgcttcggtatggatattcttctgaagcttgatttacctgctacaagaaggttctttgatgcattctttgacttagaacctcgttattggcatggcttcttatcgtctcgattgtttctacctgaactcatagtttttgggctgtctctattctctcatgcttcaaatacttctagatttgagataatgacaaagggaactgttccattagtaaatatgatcaacaatttgttacaggataaagaatgaatccgagtaattcggaatcttgtccaatctcgtgcc

SEQ ID NO: 8 - protein, 500 aa, Lycopersicon esculentum
MDTLLKTPNNLEFLNPHHGFAVKASTFRSEKHHNFGSRKFCETLGRSVCVKGSSSALLELVPETKKENLDFELPMYDPSKGVVVDLAVVGGGPAGLAVAQQVSEAGLSVCSIDPNPKLIWPNNYGVWVDEFEAMDLLDCLDATWSGAAVYIDDNTAKDLHRPYGRVNRKQLKSKMMQKCIMNGVKFHQAKVIKVIHEESKSMLICNDGITIQATVVLDATGFSRSLVQYDKPYNPGYQVAYGILAEVEEHPFDVNKMVFMDWRDSHLKNNTDLKERNSRIPTFLYAMPFSSNRIFLEETSLVARPGLRIDDIQERMVARLNHLGIKVKSIEEDEHCLIPMGGPLPVLPQRVVGIGGTAGMVHPSTGYMVARTLAAAPVVANAIIQYLGSERSHSGNELSTAVWKDLWPIERRRQREFFCFGMDILLKLDLPATRRFFDAFFDLEPRYWHGFLSSRLFLPELIVFGLSLFSHASNTSRFEIMTKGTVPLVNMINNLLQDKE

SEQ ID NO: 9 - DNA, 22 bp, Artificial Sequence
  misc_feature (1)..(22): Primer 148-Start
atgatccagttagaacaaccac

SEQ ID NO: 10 - DNA, 24 bp, Artificial Sequence
  misc_feature (1)..(24): Primer 148-End
ctattttgctttgtaaatttctgg

SEQ ID NO: 11 - DNA, 26 bp, Artificial Sequence
  misc_feature (1)..(26): Primer  38-Start
atgaatttttgtgataaaccagttag

SEQ ID NO: 12 - DNA, 23 bp, Artificial Sequence
  misc_feature (1)..(23): Primer 38-End
acgaattggttactgaattgttg

SEQ ID NO: 13 - DNA, 258 bp, Nicotiana tabacum
  transit_peptide (1)..(258): pTP09
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcgggatcc

SEQ ID NO: 14 - DNA, 260 bp, Nicotiana tabacum
  transit_peptide (1)..(260): pTP10
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcgctggatcc

SEQ ID NO: 15 - DNA, 259 bp, Nicotiana tabacum
  transit_peptide (1)..(259): pTP11
ggtaccatggcgtcttcttcttctctcactctctctcaagctatcctctctcgttctgtccctcgccatggctctgcctcttcttctcaactttccccttcttctctcactttttccggccttaaatccaatcccaatatcaccacctcccgccgccgtactccttcctccgccgccgccgccgccgtcgtaaggtcaccggcgattcgtgcctcagctgcaaccgaaaccatagagaaaactgagactgcggggatcc

(19) United States
(12) Patent Application Publication
(10) Pub. No.: US 2006/0053513 A1
(43) Pub. Date: Mar. 9, 2006

(54) Method for producing ketocarotenoids by cultivating genetically modified organisms

(76) Inventors: Sabine Steiger, Darmstadt (DE); Gerhard Sandmann, Oberusel (DE)

Correspondence Address: CONNOLLY BOVE LODGE & HUTZ, LLP, P O BOX 2207, WILMINGTON, DE, 19899, US

(21) Appl. No.: 10/541,513

(22) PCT Filed: Dec. 24, 2003
(86) PCT No.: PCT/EP03/14876

§ 371 Date: Jul. 8, 2005

(30) Foreign Application Priority Data
Jan. 9, 2003 (DE) 103-00-649.4

(51) Int. Cl. A01H 1/00; C12P 23/00; C12N 9/10

(52) U.S. Cl. 800/282; 435/67; 435/193

(57) ABSTRACT

The present invention relates to a process for preparing ketocarotenoids by cultivation of genetically modified organisms which, compared with the wild type, have a modified ketolase activity, to the genetically modified organisms, and to the use thereof as human and animal foods and for producing ketocarotenoid extracts.

A process for preparing ketocarotenoids by cultivation of genetically modified organisms

The present invention relates to a process for preparing ketocarotenoids by cultivation of genetically modified organisms which, compared with the wild type, have a modified ketolase activity, to the genetically modified organisms, and to the use thereof as human and animal foods and for producing ketocarotenoid extracts.

Ketocarotenoids occur mainly in bacteria, a few fungi and as secondary carotenoids in green algae. Besides echinenone, the 4-monoketo derivative of β-carotene, there is also formation of a corresponding symmetric diketo compound canthaxanthin. In addition, a few species are known of the abovementioned organism groups in which astaxanthin (=3,3′-dihydroxy-4,4′-diketo-β-carotene) is to be found as end product of biosynthesis (together with small amounts of corresponding intermediates) (Goodwin, T. W. (1980) The Biochemistry of the Carotenoids, Vol. 1: Plants, 2nd edn. Chapman & Hall, New York.; Johnson, E. A. & An G.-H. (1991) astaxanthin from microbial sources. Critical Rev. Biotechnol. 11, 297-326.; 3. Lorenz, R. T. & Cysewski, G. R. (2000) Commercial potential for Haematococcus microalgae as a natural source of astaxanthin. Trend Biotechn. 18, 160-167).

Because of their coloring properties, the ketocarotenoids and especially astaxanthin are used as pigmenting aids in livestock nutrition, especially in trout, salmon and shrimp rearing.

astaxanthin is currently prepared for the most part by chemical synthesis processes. Natural ketocarotenoids such as, for example, natural astaxanthin are currently obtained in small quantities in biotechnological processes by cultivation of algae, for example Haematococcus pluvialis or by fermentation of genetically optimized microorganisms and subsequent isolation.

An economic biotechnological process for preparing natural ketocarotenoids is therefore of great importance.

Specific ketolase genes of the crtW type have been cloned and functionally identified from the bacteria Agrobacterium aurantiacum (EP 735 137, Accession No. D58420), Paracoccus marcusii (Accession No. Y15112) and as cDNA from Haematococcus (Haematococcus pluvialis Flotow em. Wille and Haematoccus pluvialis, NIES-144 (EP 725137, WO 98/18910 and Lotan et al, FEBS Letters 1995, 364,125-128, Accession No. X86782 and D45881)).

There also exist ORFs from other organisms which, because of amino acid homologies, are referred to as ketolase genes, such as, for example, nucleic acids encoding a ketolase from Alcaligenes sp. PC-1 (EP 735137, Accession No. D58422), Synechocystis sp. strain PC6803 (Accession No. NP_{—}442491), Bradyrhizobium sp. (Accession No. AF218415), Nostoc sp. PCC 7120 (Kaneko et al, DNA Res. 2001, 8(5), 205-213; Accession No. AP003592, BAB74888) and Brevundimonas aurantiaca (WO 02079395).

Only the ketolases from A. aurantiacum and Alcaligenes spec. have been biochemically characterized (Fraser P. D., Shimada H. & Misawa N. (1998) Enzymic confirmation of reactions involved in routes to astaxanthin formation, elucidated using a direct substrate in vitro assay. Eur. J. Biochem. 252, 229-236.). There is a further type of 1-carotene ketolase genes, crtO from the cyanobacterium Synechocystis, which has no similarity with crtW and is related to the bacterial desaturases (Femandez-Gonzalez, B., Sandmann, G. & Vioque, A (1997) A new type of asymmetrically acting 1-carotene ketolase is required for the synthesis of echinenone in the cyanobacterium Synechocystis sp. PCC 6803. J. Biol. Chem. 272, 9728-9733.)

All known ketolases are able to introduce a keto group in position 4 of β-carotene. The crtO gene codes for a monoketolase which forms echinenone as end product from β-carotene. The crtW gene family, to which bkt from Haematococcus also belongs, codes for a diketolase which converts β-carotene as far as canthaxanthin. This reaction appears to be the first modification step in the direction of astaxanthin, which is followed by a hydroxylation at position 3. The same reaction sequence then also applies to the second ionone ring (9). There is also enzymatic evidence that 3-hydroxy-β-carotene derivatives can be ketonized only poorly at position 4. It has likewise emerged that only certain bacterial hydroxylases, such as those from Erwinia uredovora (Breitenbach, J., Misawa, N., Kajiwara, S. & Sandmann, G. (1996) Expression in Escherichia coli and properties of the carotene ketolase from Haematococcus pluvialis. FEMS Microbiol. Left. 140, 241-246) or A. aurantiacum, are able to convert ketonized intermediates. The structurally different hydroxylases of cyanobacteria are not capable of this (Albrecht, M., Steiger, S. & Sandmann, G. (2001) Expression of a ketolase gene mediates the synthesis of canthaxanthin in Synechococcus leading to resistance against pigment photodegradation and UV-B sensitivity of photosynthesis. Photochem. Photobiol. 73, 551-555). There is no cooperation of this type of hydroxylase with a ketolase, and no substantial quantities of astaxanthin are obtained.

EP 735 137 describes the preparation of xanthophylls in microorganisms such as, for example, E. coli by introducing ketolase genes (crtw) from Agrobacterium aurantiacum or Alcaligenes sp. PC-1 into microorganisms.

EP 725 137, WO 98/18910, Kajiwara et al. (Plant Mol. Biol. 1995, 29, 343-352) and Hirschberg et al. (FEBS Letters 1995, 364, 125-128) disclose the preparation of astaxanthin by introducing ketolase genes from Haematococcus pluvialis (crtW, crtO or bkt) into E. coli.

Hirschberg et al. (FEBS Letters 1997, 404, 129-134) describe the preparation of astaxanthin in Synechococcus by introducing ketolase genes (crtO) from Haematococcus pluvialis. Sandmann et al. (Photochemistry and Photobiology 2001, 73(5), 551-55) describe an analogous process which, however, leads to the preparation of canthaxanthin and provides only traces of astaxanthin.

WO 98/18910 and Hirschberg et al. (Nature Biotechnology 2000, 18(8), 888-892) describe the synthesis of ketocarotenoids in nectaries of tobacco flowers by introducing the ketolase gene from Haematococcus pluvialis (crtO) into tobacco.

WO 01/20011 describes a DNA construct for producing ketocarotenoids, especially astaxanthin, in seeds of oilseed crops such as rape, sunflower, soybean and mustard, using a seed-specific promoter and a ketolase from Haematococcus pluvialis.

All the processes described in the prior art for preparing ketocarotenoids and, in particular, the processes described for preparing astaxanthin have the disadvantage that the transgenic organisms provide only small quantities of astaxanthin.

It is an object of the present invention to provide a process for preparing ketocarotenoids by cultivation of genetically modified organisms, and to provide further genetically modified organisms which produce ketocarotenoids, which have the prior art disadvantages described above to a smaller extent or not at all.

We have found that this object is achieved by a process for preparing ketocarotenoids by cultivating genetically modified organisms which, compared with the wild type, have a modified ketolase activity, and the modified ketolase activity is caused by a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

The organisms of the invention, such as, for example, microorganisms or plants, are preferably able as starting organisms naturally to produce carotenoids such as, for example, β-carotene or zeaxanthin, or can be made able by genetic modification such as, for example, reregulation of metabolic pathways or complementation to produce carotenoids such as, for example, β-carotene or zeaxanthin.

Some organisms are already able as starting or wild-type organisms to produce ketocarotenoids such as, for example, astaxanthin or canthaxanthin. These organisms, such as, for example, Haematococcus pluvialis, Paracoccus marcusil, Xanthophyllomyces dendrorhous, Bacillus circulans, Chlorococcum, Phaffia rhodozyma, Adonis sp., Neochloris wimmeri, Protosiphon botryoides, Scotiellopsis oocystiformis, Scenedesmus vacuolatus, Chlorela zofingiensis, Ankistrodesmus braunii, Euglena sanguinea, Bacillus atrophaeus, Blakeslea already have as starting or wild-type organism a ketolase activity.

In one embodiment of the process of the invention, therefore, the starting organisms used are those already having a ketolase activity as wild type or starting organism. In this embodiment, the genetic modification brings about an increase in the ketolase activity compared with the wild type or starting organism.

Ketolase activity means the enzymic activity of a ketolase.

A ketolase means a protein which has the enzymatic activity of introducing a keto group on the, optionally substituted, β-ionone ring of carotenoids.

A ketolase means in particular a protein having the enzymatic activity of converting β-carotene into canthaxanthin.

Accordingly, ketolase activity means the amount of β-carotene converted or amount of canthaxanthin produced in a particular time by the ketolase protein.

Thus, when a ketolase activity is increased compared with the wild type, the amount of β-carotene converted or the amount of canthaxanthin produced in a particular time is increased by the ketolase protein compared with the wild type.

This increase in the ketolase activity is preferably at least 5%, more preferably at least 20%, more preferably at least 50%, more preferably at least 100%, preferably at least 300%, more preferably at least 500%, in particular at least 600%, of the ketolase acitivty of the wild type.

The term “wild type” means according to the invention the corresponding starting organism.

Depending on the context, the term “organism” may mean the starting organism (wild type) or a genetically modified organism of the invention, or both.

“Wild type” means, preferably and especially in cases where the organism or the wild type cannot be unambiguously assigned, in each case a reference organism for the increasing or causing of the ketolase activity, for the increasing, described hereinafter, of the hydroxylase activity, for the increasing, described hereinafter, of the β-cyclase activity and the increasing of the content of ketocarotenoids.

This reference organism for microorganisms which already have a ketolase activity as wild type is preferably Haematococcus pluvialis.

This reference organism for microorganisms which have no ketolase activity as wild type is preferably Blakeslea.

This reference organism for plants which already have a ketolase activity as wild type is preferably Adonis aestivalis, Adonis flammeus or Adonis annuus, particularly preferably Adonis aestivalis.

This reference organism for plants which have no ketolase activity in petals as wild type is preferably Tagetes erecta, Tagetes patula, Tagetes lucida, Tagetes pringlei, Tagetes palmeri, Tagetes minuta or Tagetes campanulata, particularly preferably Tagetes erecta.

Determination of the ketolase activity in the genetically modified organisms of the invention and in wild-type and reference organisms preferably takes place under the following conditions:

Determination of the ketolase activity in plant or microorganism material is based on the method of Frazer et al., (J. Biol. Chem. 272(10): 6128-6135, 1997). The ketolase activity in plant or microorganism extracts is determined using the substrates β-carotene and canthaxanthin in the presence of lipid (soybean lecithin) and detergent (sodium cholate). Substrate/product ratios from ketolase assays are measured by means of HPLC.

Various ways are possible for increasing the ketolase activity, for example by switching off inhibitory regulatory mechanisms at the translation and protein level or by increasing the gene expression of a nucleic acid encoding a ketolase compared with the wild type, for example by inducing the ketolase gene by activators or by introducing nucleic acids encoding a ketolase into the organism.

Increasing the gene expression of a nucleic acid encoding a ketolase also means according to the invention in this embodiment the manipulation of the expression of the organisms own endogenous ketolases. This can be achieved for example by modifying the promoter DNA sequence for ketolase-encoding genes. Such a modification, which results in a modified or, preferably, increased expression rate of at least one endogenous ketolase gene, can also be effected by deletion or insertion of DNA sequences.

It is possible as described above to modify the expression of at least one endogenous ketolase through application of exogenous stimuli. This can be effected by particular physiological conditions, i.e. through application of foreign substances.

A further possibility for achieving an increased expression of at least one endogenous ketolase gene is for a regulator protein which does not occur in the wild-type organism or is modified to interact with the promoter of these genes.

A regulator of this type may be a chimeric protein which consists of a DNA-binding domain and of a transcription activator domain as described, for example, in WO 96/06166.

In a preferred embodiment, the ketolase activity is increased by comparison with the wild type by increasing the gene expression of a nucleic acid encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

In a further preferred embodiment, the gene expression of a nucleic acid encoding a ketolase is increased by introducing nucleic acids which encode ketolases, where the ketolases have the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, into the organisms.

Thus, in this embodiment, at least one further ketolase gene encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, is present in the transgenic organisms of the invention compared with the wild type.

In this embodiment, the genetically modified organism of the invention accordingly has at least one exogenous (=heterologous) nucleic acid encoding a ketolase, or has at least two endogenous nucleic acids encoding a ketolase, where the ketolases comprise the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

In another, preferred embodiment of the process of the invention, the organisms used as starting organisms have no ketolase activity as wild type.

In this preferred embodiment, the genetic modification causes the ketolase activity in the organisms. The genetically modified organism of the invention thus has in this preferred embodiment a ketolase activity compared with the genetically unmodified wild type, and is thus preferably capable of transgenic expression of a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

In this preferred embodiment, the gene expression of a nucleic acid encoding a ketolase is caused, in analogy to the increasing, described above, of the gene expression of a nucleic acid encoding a ketolase, preferably by introducing nucleic acids which encode ketolases comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, into the starting organism.

It is possible to use for this purpose in both embodiments in principle all nucleic acids which encode a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

The use of the nucleic acids of the invention encoding a ketolase leads in the process of the invention surprisingly to a higher yield of ketocarotenoids, especially of astaxanthin, than on use of the ketolase genes used in the prior art.

All the nucleic acids mentioned in the description may be, for example, an RNA, DNA or cDNA sequence.

In the case of genomic ketolase sequences from eukaryotic sources which comprise introns, it is preferred to use nucleic acid sequences which have already been processed, such as the corresponding cDNAs, in the case where the host organism is unable or cannot be made able to express the corresponding ketolase.

Examples of nucleic acids encoding a ketolase, and the corresponding ketolases comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, which can be used advantageously in the process of the invention are, for example, sequences from

Nostoc punctiforme PCC73102 ORF 38, nucleic acid: Acc. No. NZ_AABC01000195, base pair 55,604 to 55,392 (SEQ ID NO: 1); protein: Acc. No. ZP_{—}00111258 (SEQ ID NO: 2) (annotated as putative protein) or

Nostoc punctiforme PCC73102 ORF 148, nucleic acid: Acc. No. NZ_AABC01000196, base pair 140,571 to 139,810 (SEQ ID NO: 3), protein: (SEQ ID NO: 4) (not annotated) or ketolase sequences derived from these sequences.

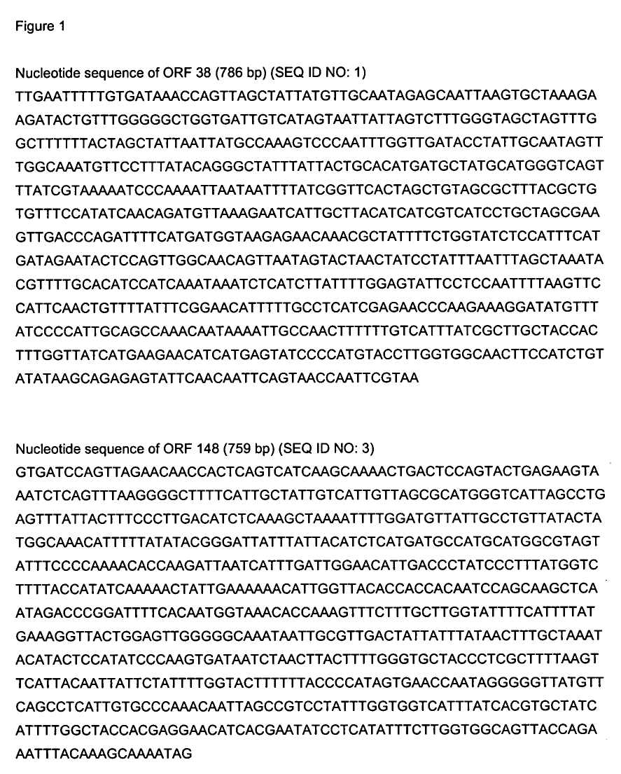

FIG. 1 shows additionally the nucleic acid sequences of ORF 38 and ORF 148 from Nostoc punctiforme.

For the preparation of astaxanthin it is particularly preferred to use in particular the ketolase of Nostoc punctiforme PCC73102 ORF 148, nucleic acid: Acc. No. NZ_AABC01000196, base pair 140,571 to 139,810 (SEQ ID NO: 3), protein: (SEQ ID NO: 4) or sequences derived from this sequence.

Further natural examples of ketolases and ketolase genes which can be used in the process of the invention can easily be found for example from various organisms whose genomic sequence is known through identity comparisons of the amino acid sequences or of the corresponding back-translated nucleic acid sequences from databases with the sequences SEQ ID NO: 2 or SEQ ID NO: 4 described above.

Further natural examples of ketolases and ketolase genes can additionally be easily found starting from the nucleic acid sequences above, in particular starting from the sequences SEQ ID NO: 1 or SEQ ID NO: 3 from various organisms whose genomic sequence is unknown through hybridization techniques in a manner known per se.

The hybridization can take place under moderate (low stringency) or preferably under stringent (high stringency) conditions.

Hybridization conditions of these types are described for example in Sambrook, J., Fritsch, E. F., Maniatis, T., in: Molecular Cloning (A Laboratory Manual), 2nd edition, Cold Spring Harbor Laboratory Press, 1989, pages 9.31-9.57 or in Current Protocols in Molecular Biology, John Wiley & Sons, N.Y. (1989), 6.3.1-6.3.6.

For example, the conditions during the washing step can be selected from the range of conditions limited by those of low stringency (with 2×SSC at 50° C.) and those of high stringency (with 0.2×SSC at 50° C., preferably at 65° C.) (20×SSC: 0.3 M sodium citrate, 3 M sodium chloride, pH 7.0).

An additional possibility is to raise the temperature during the washing step from moderate conditions at room temperature, 22° C., up to stringent conditions at 65° C.

Both parameters, the salt concentration and temperature, can be varied simultaneously, and it is also possible to keep one of the two parameters constant and vary only the other one. It is also possible to employ denaturing agents such as, for example, formamide or SDS during the hybridization. Hybridization in the presence of 50% formamide is preferably carried out at 42° C.

Some examples of conditions for hybridization and washing step are given below:

(1) Hybridization Conditions with for Example

(i) 4×SSC at 65° C., or
(ii) 6×SSC at 45° C., or
(iii) 6×SSC at 68° C., 100 mg/ml denatured fish sperm DNA, or
(iv) 6×SSC, 0.5% SDS, 100 mg/ml denatured, fragmented salmon sperm DNA at 68° C., or
(v) 6×SSC, 0.5% SDS, 100 mg/ml denatured, fragmented salmon sperm DNA, 50% formamide at 42° C., or
(vi) 50% formamide, 4×SSC at 42° C., or
(vii) 50% (vol/vol) formamide, 0.1% bovine serum albumin, 0.1% Ficoll, 0.1% polyvinylpyrrolidone, 50 mM sodium phosphate buffer pH 6.5, 750 mM NaCl, 75 mM sodium citrate at 42° C., or
(viii) 2× or 4×SSC at 50° C. (moderate conditions), or
(ix) 30 to 40% formamide, 2× or 4×SSC at 42° C. (moderate conditions).
   (2) Washing Step for 10 Minutes Each with for Example
(i) 0.015 M NaCl/0.0015 M sodium citrate/0.1% SDS at 50° C., or
(ii) 0.1×SSC at 65° C., or
(iii) 0.1×SSC, 0.5% SDS at 68° C., or
(iv) 0.1×SSC, 0.5% SDS, 50% formamide at 42° C., or
(v) 0.2×SSC, 0.1% SDS at 42° C., or
(vi) 2×SSC at 65° C. (moderate conditions).

In a preferred embodiment of the process of the invention there is introduction of nucleic acids which encode a ketolase comprising the amino acid sequence SEQ ID NO: 2 or a sequence which is derived from this sequence by substitution, insertion or deletion of amino acids and which has an identity of at least 50%, preferably at least 60%, preferably at least 65%, preferably at least 70%, more preferably at least 75%, more preferably at least 80%, more preferably at least 85%, more preferably at least 90%, more preferably at least 95%, particularly preferably at least 98%, at the amino acid level with the sequence SEQ ID NO: 2.

It is moreover possible for the ketolase sequence to be a natural one which can be found as described above by identity comparison of the sequences from other organisms, or for the ketolase sequence to be an artificial one which has been modified starting from the sequence SEQ ID NO: 2 by artificial variation, for example by substitution, insertion or deletion of amino acids.

The term “substitution” means in the description substitution of one or more amino acids by one or more amino acids. So-called conservative substitutions are preferably carried out, in which the replaced amino acid has a similar property to the original amino acid, for example substitution of Glu by Asp, Gln by Asn, Val by lie, Leu by lIe, Ser by Thr.

Deletion is the replacement of an amino acid by a direct linkage. Preferred positions for deletions are the termini of the polypeptide and the linkages between the individual protein domains.

Insertions are introductions of amino acids into the polypeptide chain, with formal replacement of a direct linkage by one or more amino acids.

Identity between two proteins means the identity of the amino acids over the entire length of each protein, in particular the identity calculated by comparison using the vector NTI suite 7.1 software supplied by Informax (USA) using the clustal method (Higgins D G, Sharp P M. Fast and sensitive multiple sequence alignments on a microcomputer. Comput Appl. Biosci. 1989 Apr.; 5(2):151-1), setting the following parameters:

Multiple Alignment Parameter:

| | |
|---|---|
| Gap opening penalty | 10 |
| Gap extension penalty | 10 |
| Gap separation penalty range | 8 |
| Gap separation penalty | off |
| % identity for alignment delay | 40 |
| Residue specific gaps | off |
| Hydrophilic residue gap | off |
| Transition weight | 0 |

Pairwise Alignment Parameter:

FAST algorithm on

| | |
|---|---|
| K-tuple size | 1 |
| Gap penalty | 3 |
| Window size | 5 |
| Number of best diagonals | 5 |

The ketolase having an identity of at least 42% at the amino acid level with the sequence SEQ ID NO: 2 accordingly means a ketolase which, on comparison of its sequence with the sequence SEQ ID NO: 2, in particular using the above program algorithm with the above set of parameters, has an identity of at least 42%.

For example, using the above program algorithm with the above set of parameters, the sequence of the ketolase from Nostoc punctiforme PCC73102 ORF 148 (SEQ ID NO: 4) shows an identity of 64% with the sequence of the ketolase from Nostoc punctiforme PCC73102 ORF 38 (SEQ ID NO: 2).

Suitable nucleic acid sequences can be obtained for example by back-translation of the polypeptide sequence in accordance with the genetic code.

The codons preferably used for this purpose are those frequently used in accordance with the organism-specific codon usage. The codon usage can easily be found by means of computer analyses of other, known genes in the relevant organisms.

In a particularly preferred embodiment, a nucleic acid comprising the sequence SEQ ID NO: 1 or SEQ ID NO: 3 is introduced into the organism.

All the aforementioned ketolase genes can moreover be prepared in a manner known per se by chemical synthesis from the nucleotide units such as, for example, by fragment condensation of individual overlapping, complementary nucleic acid units of the double helix. Chemical synthesis of oligonucleotides is possible, for example, in a known manner by the phosphoramidite method (Voet, Voet, 2nd edition, Wiley Press New York, pages 896-897). Addition of synthetic oligonucleotides and filling in of gaps using the Klenow fragment of DNA polymerase and ligation reactions, and general cloning methods, are described in Sambrook et al. (1989), Molecular cloning: A laboratory manual, Cold Spring Harbor Laboratory Press.

The identity shown by the sequence of the ketolase from Nostoc punctiforme PCC73102 ORF 38 (SEQ ID NO: 2) with the sequences of the ketolases used in the prior art processes is 38% (Agrobacterium aurantiacum (EP 735 137), Accession No. D58420), 38% (Alcaligenes sp. PC-1 (EP 735137), Accession No. D58422) and 19 to 21% (Haematococcus pluvialis Flotow em. Wille and Haematoccus pluvialis, NIES 144 (EP 725137, WO 98/18910 and Lotan et al, FEBS Letters 1995, 364, 125 128), Accession No. X86782 and D45881).

In a preferred embodiment, organisms which have an increased hydroxylase activity and/or β-cyclase activity in addition to the increased ketolase activity compared with the wild type are cultivated.

Hydroxylase activity means the enzymic activity of a hydroxylase.

A hydroxylase means a protein having the enzymatic activity of introducing a hydroxyl group on the, optionally substituted, β-ionone ring of carotenoids.

In particular, a hydroxylase means a protein having the enzymatic activity of converting β-carotene into zeaxanthin or canthaxanthin into astaxanthin.

Accordingly, hydroxylase activity means the amount of β-carotene or canthaxanthin converted, or amount of zeaxanthin or astaxanthin produced, by the hydroxylase protein.

Thus, when the hydroxylase activity is increased compared with the wild type, the amount of β-carotene or canthaxantin converted or the amount of zeaxanthin or astaxanthin produced in a particular time by the hydroxylase protein is increased compared with the wild type.

This increase in the hydroxylase activity is preferably at least 5%, further preferably at least 20%, further preferably at least 50%, further preferably at least 100%, more preferably at least 300%, even more preferably at least 500%, in particular at least 600%, of the hydroxylase activity of the wild type.

β-Cyclase activity means the enzymic activity of a β-cyclase.

A β-cyclase means a protein having the enzymatic activity of converting a terminal linear lycopene residue into a β-ionone ring.

In particular, a β-cyclase means a protein having the enzymatic activity of converting γ-carotene into β-carotene.

Accordingly, a β-cyclase activity means the amount of γ-carotene converted or the amount of β-carotene produced in a particular time by the β-cyclase protein.

Thus, when the β-cyclase activity is increased compared with the wild type, the amount of lycopene or γ-carotene converted or the amount of γ-carotene produced from lycopene or the amount of β-carotene produced from carotene by the β-cyclase protein in a particular time is increased compared with the wild type.

This increase in the β-cyclase activity is preferably at least 5%, further preferably at least 20%, further preferably at least 50%, further preferably at least 100%, more preferably at least 300%, even more preferably at least 500%, in particular at least 600%, of the β-cyclase activity of the wild type.

The hydroxylase activity in the genetically modified organisms of the invention and in wild-type and reference organisms is preferably determined under the following conditions:

The hydroxylase activity is determined by the method of Bouvier et al. (Biochim. Biophys. Acta 1391 (1998), 320-328) in vitro. Ferredoxin, ferredoxin-NADP⁺ oxidoreductase, catalase, NADPH and β-carotene with mono- and digalactosyl glycerides are added to a defined amount of organism extract.

The hydroxylase activity is particularly preferably determined under the following conditions of Bouvier, Keller, d'Harlingue and Camara (Xanthophyll biosynthesis: molecular and functional characterization of carotenoid hydroxylases from pepper fruits (Capsicum annuum L.); Biochim. Biophys. Acta 1391 (1998), 320-328):

The in vitro assay is carried out in a volume of 0.250 ml. The mixture contains 50 mM potassium phosphate (pH 7.6), 0.025 mg of spinach ferredoxin, 0.5 units of spinach ferredoxin-NADP⁺ oxidoreductase, 0.25 mM NADPH, 0.010 mg of beta-carotene (emulsified in 0.1 mg of Tween 80), 0.05 mM of a mixture of mono- and digalactosyl glycerides (1:1), 1 unit of catalyse, 0.2 mg of bovine serum albumin and organism extract in a different volume. The reaction mixture is incubated at 30° C. for 2 hours. The reaction products are extracted with organic solvents such as acetone or chloroform/methanol (2:1) and determined by HPLC.

The β-cyclase activity in the genetically modified organisms of the invention and in wild-type and reference organisms is preferably determined under the following conditions:

The β-cyclase activity is determined by the method of Fraser and Sandmann (Biochem. Biophys. Res. Comm. 185(1) (1992) 9 15) in vitro. Potassium phosphate is added as buffer (pH 7.6), lycopene as substrate, paprika stromal protein, NADP⁺, NADPH and ATP to a defined amount of organism extract.

The β-cyclase activity is particularly preferably determined under the following conditions of Bouvier, d'Harlingue and Camara (Molecular Analysis of carotenoid cyclase inhibition; Arch. Biochem. Biophys. 346(1) (1997) 53-64):

The in vitro assay is carried out in a volume of 250 μl. The mixture contains 50 mM potassium phosphate (pH 7.6), various amounts of organism extract, 20 nM lycopene, 250 μg of paprika chromoplastid stromal protein, 0.2 mM NADP⁺, 0.2 mM NADPH and 1 mM ATP. NADP/NADPH and ATP are dissolved in 10 ml of ethanol with 1 mg of Tween 80 immediately before addition to the incubation medium. After a reaction time of 60 minutes at 30° C., the reaction is stopped by adding chloroform/methanol (2:1). The reaction products extracted into chloroform are analyzed by HPLC.

An alternative assay with radioactive substrate is described in Fraser and Sandmann (Biochem. Biophys. Res. Comm. 185(1) (1992) 9-15).

The hydroxylase activity and/or β-cyclase activity can be increased in various ways, for example by switching off inhibitory regulatory mechanisms at the expression and protein level or by increasing the gene expression of nucleic acids encoding a hydroxylase, and/or of nucleic acids encoding a β-cyclase, compared with the wild type.

The gene expression of nucleic acids encoding a hydroxylase, and/or the gene expression of the nucleic acid encoding a β-cyclase, compared with the wild type, can likewise be increased in various ways, for example by inducing the hydroxylase gene and/or β-cyclase gene by activators or by introducing one or more hydroxylase gene copies and/or β-cyclase gene copies, i.e. by introducing at least one nucleic acid encoding a hydroxylase, and/or at least one nucleic acid encoding a β-cyclase, into the organism.

Increasing the gene expression of a nucleic acid encoding a hydroxylase and/or β-cyclase also means according to the invention manipulation of the expression of the organism's own endogenous hydroxylase and/or β-cyclase.

This can be achieved for example by modifying the promoter DNA sequence for genes encoding hydroxylases and/or β-cyclases. Such a modification, resulting in an increased expression rate of the gene, can be effected for example by deletion or insertion of DNA sequences.

It is possible, as described above, to modify the expression of the endogenous hydroxylase and/or β-cyclase by application of exogenous stimuli. This can be effected by particular physiological conditions, i.e. by application of foreign substances.

A further possibility for achieving a modified or increased expression of an endogenous hydroxylase and/or β-cyclase gene is through interaction of a regulator protein which does not occur in the untransformed organism with the promoter of this gene.

Such a regulator may be a chimeric protein consisting of a DNA-binding domain and of a transcription activator domain as described, for example, in WO 96/06166.

In a preferred embodiment, the gene expression of a nucleic acid encoding a hydroxylase, and/or the gene expression of a nucleic acid encoding a β-cyclase, is increased by introducing at least one nucleic acid encoding a hydroxylase, and/or by introducing at least one nucleic acid encoding a β-cyclase, into the organism.

It is possible to use for this purpose in principle any hydroxylase gene or any β-cyclase gene, i.e. any nucleic acid which encodes a hydroxylase and any nucleic acid which encodes a β-cyclase.

In the case of genomic hydroxylase or β-cyclase nucleic acid sequences from eukaryotic sources which comprise introns, it is preferred to use nucleic acid sequences which have already been processed, such as the corresponding cDNAs, in the case where the host organism is unable or cannot be made able to express the corresponding hydroxylase or β-cyclase.

One example of a hydroxylase gene is a nucleic acid encoding a hydroxylase from Haematococcus pluvialis (Accession AX038729, WO 0061764); (nucleic acid: SEQ ID NO: 5, protein: SEQ ID NO: 6).

One example of a β-cyclase gene is a nucleic acid encoding a α-cyclase from tomato (Accession X86452) (nucleic acid: SEQ ID NO: 7, protein: SEQ ID NO: 8).

Thus, in this preferred embodiment, at least one further hydroxylase gene and/or β-cyclase gene is present in the preferred transgenic organisms of the invention compared with the wild type.

In this preferred embodiment, the genetically modified organism has for example at least one exogenous nucleic acid encoding a hydroxylase, or at least two endogenous nucleic acids encoding a hydroxylase and/or at least one exogenous nucleic acid encoding a β-cyclase, or at least two endogenous nucleic acids encoding a α-cyclase.

The hydroxylase genes preferably used in the preferred embodiment described above are nucleic acids encoding proteins comprising the amino acid sequence SEQ ID NO: 6 or a sequence which is derived from this sequence by substitution, insertion or deletion of amino acids and which have an identity of at least 30%, preferably at least 50%, more preferably at least 70%, even more preferably at least 90%, most preferably at least 95%, at the amino acid level with the sequence SEQ ID NO: 6, and which have the enzymatic property of a hydroxylase.

Further examples of hydroxylases and hydroxylase genes can be easily found for example from various organisms whose genomic sequence is known as described above by homology comparisons of the amino acid sequences or of the corresponding back-translated nucleic acid sequences from databases with SEQ ID. NO: 6.

Further examples of hydroxylases and hydroxylase genes can easily be found in a manner known per se in addition for example starting from the sequence SEQ ID NO: 5 from various organisms whose genomic sequence is unknown, as described above, by hybridization and PCR techniques.

In a further particularly preferred embodiment, nucleic acids which encode proteins comprising the amino acid sequence of the hydroxylase of the sequence SEQ ID NO: 6 are introduced into organisms to increase the hydroxylase activity.

Suitable nucleic acid sequences can be obtained for example by back-translation of the polypeptide sequence in accordance with the genetic code.

The codons used for this purpose are preferably those frequently used in accordance with the organism-specific codon usage. This codon usage can easily be found by means of computer analyses of other, known genes of the relevant organisms.

In a particularly preferred embodiment, a nucleic acid comprising the sequence SEQ. ID. NO: 5 is introduced into the organism.

The β-cyclase genes preferably used in the preferred embodiment described above are nucleic acids which encode proteins comprising the amino acid sequence SEQ ID NO: 8 or a sequence which is derived from this sequence by substitution, insertion or deletion of amino acids and which has an identity of at least 30%, preferably at least 50%, more preferably at least 70%, even more preferably at least 90%, most preferably at least 95%, at the amino acid level with the sequence SEQ ID NO: 8, and which has the enzymatic property of a β-cyclase.

Further examples of β-cyclases and β-cyclase genes can easily be found for example from various organisms whose genomic sequence is known as described above by homology comparisons of the amino acid sequences or of the corresponding back-translated nucleic acid sequences from databases with the SEQ ID NO: 8.

Further examples of β-cyclases and β-cyclase genes can easily be found in a manner known per se in addition for example starting from the sequence SEQ ID NO: 7 from various organisms whose genomic sequence is unknown by hybridization and PCR techniques.

In a further particularly preferred embodiment, nucleic acids which encode proteins comprising the amino acid sequence of β-cyclase of the sequence SEQ. ID. NO: 8 are introduced into organisms to increase the β-cyclase activity.

Suitable nucleic acid sequences can be obtained for example by back-translation of the polypeptide sequence in accordance with the genetic code.

The codons preferably used for this purpose are those frequently used in accordance with the organ-specific codon usage. This codon usage can easily be found by means of computer analyses of other, known genes of the relevant organisms.

In a particularly preferred embodiment, a nucleic acid comprising the sequence SEQ. ID. NO: 7 is introduced into the organism.

All the aforementioned hydroxylase genes or β-cyclase genes can moreover be prepared in a manner known per se by chemical synthesis from the nucleotide units such as, for example, by fragment condensation of individual overlapping, complementary nucleic acid units of the double helix. Chemical synthesis of oligonucleotides is possible, for example, in a known manner by the phosphoramidite method (Voet, Voet, 2nd edition, Wiley Press New York, pages 896-897). Addition of synthetic oligonucleotides and filling in of gaps using the Klenow fragment of DNA polymerase and ligation reactions, and general cloning methods, are described in Sambrook et al. (1989), Molecular cloning: A laboratory manual, Cold Spring Harbor Laboratory Press.

The genetically modified organisms particularly preferably used in the process of the invention have the following combinations of genetic modifications:
genetically modified organisms which have, compared with the wild type, an increased or caused ketolase activity and an increased hydroxylase activity,
genetically modified organisms which have, compared with the wild type, an increased or caused ketolase activity and an increased β-cyclase activity and genetically modified organisms which have, compared with the wild type, an increased or caused ketolase activity and an increased hydroxylase activity and an increased β-cyclase activity.

These genetically modified organisms can be produced as described hereinafter for example by introducing individual nucleic acid constructs (expression cassettes) or by introducing multiple constructs which comprise up to two or three of the described activities.

Organisms preferably mean according to the invention organisms which are able as wild-type or starting organisms naturally or through genetic complementation and/or reregulation of metabolic pathways to produce carotenoids, in particular β-carotene and/or zeaxanthin and/or neoxanthin and/or violaxanthin and/or lutein.

Further preferred organisms already have as wild-type or starting organisms a hydroxylase activity and are thus able as wild-type or starting organisms to produce zeaxanthin.

Preferred organisms are plants or microorganisms such as, for example, bacteria, yeasts, algae or fungi.

Bacteria which can be used are both bacteria which are able, because of the introduction of genes of carotenoid biosynthesis of a carotenoid-producing organism, to synthesize xanthophylls, such as, for example, bacteria of the genus Escherichia, which comprise for example crt genes from Erwinia, and bacteria which are intrinsically able to synthesize xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobacterium sp. strain R1534, the cyanobacterium Synechocystis sp. PCC6803, Paracoccus marcusii or Paracoccus carotinifaciens.

Preferred yeasts are Candida, Saccharomyces, Hansenula, Pichia or Phaffia. Particularly preferred yeasts are Xanthophyllomyces dendrorhous or Phaffia rhodozyma.

Preferred fungi are Aspergillus, Trichoderma, Ashbya, Neurospora, Blakeslea, Phycomyces, Fusarium or other fungi described in Indian Chem. Engr. Section B. Vol. 37, No. 1, 2 (1995) on page 15, table 6.

Preferred algae are green algae such as, for example, algae of the genus Haematococcus, Phaedactylum tricornatum, Volvox or Dunaliella. Particularly preferred algae are Haematococcus pluvialis or Dunaliella bardawil.

Further microorganisms which can be used and the production thereof for carrying out the process of the invention are disclosed for example in DE-A-199 16 140, which is incorporated herein by reference.

Particularly preferred plants are plants selected from the families Ranunculaceae, Berberidaceae, Papaveraceae, Cannabaceae, Rosaceae, Fabaceae, Linaceae, Vitaceae, Brassicaceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, llliaceae or Lamiaceae.

Very particularly preferred plants are selected from the group of plant genera Marigold, Tagetes erecta, Tagetes patula, Acacia, Aconitum, Adonis, Amica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hibiscus, Heliopsis, Hypericum, Hypochoeris, Impatiens, Iris, Jacaranda, Kenia, Labumum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group of plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

In the process of the invention for preparing ketocarotenoids, the step of cultivating the genetically modified organisms is preferably followed by a harvesting of the organisms and further preferably in addition by an isolation of ketocarotenoids from the organisms.

The harvesting of the organisms takes place in a manner known per se appropriate for the particular organism. Microorganisms such as bacteria, yeasts, algae or fungi or plant cells cultivated by fermentation in liquid nutrient media can be removed for example by centrifugation, decantation or filtration. Plants are grown on nutrient media and appropriately harvested in a manner known per se.

The genetically modified microorganisms are preferably cultivated in the presence of oxygen at a cultivation temperature of at least about 20° C., such as for example, 20° C. to 40° C., and at a pH of about 6 to 9. In the case of genetically modified microorganisms, the microorganisms are preferably initially cultivated in the presence of oxygen and in a complex medium such as, for example, TB or LB medium at a cultivation temperature of about 20° C. or more, and at a pH of about 6 to 9, until a sufficient cell density is reached. In order to be able to control the oxidation reaction better, it is preferred to use an inducible promoter. The cultivation is continued after induction of ketolase expression in the presence of oxygen for example for 12 hours to 3 days.

The ketocarotenoids are isolated from the harvested biomass in a manner known per se, for example by extraction and, where appropriate, further chemical or physical purification processes such as, for example, precipitation methods, crystallography, thermal separation processes, such as rectification processes or physical separation processes such as, for example, chromatography.

As mentioned below, the ketocarotenoids can be specifically produced in the genetically modified plants of the invention preferably in various plant tissues such as, for example, seeds, leaves, fruits, flowers, especially in petals.

Ketocarotenoids are isolated from the harvested petals in a manner known per se, for example by drying and subsequent extraction and, where appropriate, further chemical or physical purification processes such as, for example, precipitation methods, crystallography, thermal separation processes such as rectification processes or physical separation processes such as, for example, chromatography. Ketocarotenoids are isolated from petals for example preferably by organic solvents such as acetone, hexane, ether or methyl tert-butyl ether.

Further processes for isolating ketocarotenoids, especially from petals, are described for example in Egger and Kleinig (Phytochemistry (1967) 6, 437-440) and Egger (Phytochemistry (1965) 4, 609-618).

The ketocarotenoids are preferably selected from the group of astaxanthin, canthaxanthin, echinenone, 3-hydroxyechinenone, 3′-hydroxyechinenone, adonirubin and adonixanthin.

astaxanthin is a particularly preferred ketocarotenoid.

Depending on the organism used, the ketocarotenoids are obtained in free form or as fatty acid ester.

In plant petals, the ketocarotenoids are obtained in the process of the invention in the form of their mono- or diesters with fatty acids. Some examples of detected fatty acids are myristic acid, palmitic acid, stearic acid, oleic acid, linolenic acid and lauric acid (Kamata and Simpson (1987) Comp. Biochem. Physiol. Vol. 86B(3), 587-591).

The ketocarotenoids can be produced in the whole plant or, in a preferred embodiment, specifically in plant tissues containing chromoplasts. Examples of preferred plant tissues are roots, seeds, leaves, fruits, flowers and, in particular nectaries and petals.

In a particularly preferred embodiment of the process of the invention, genetically modified plants which show the highest rate of expression of a ketolase in flowers are used.

This is preferably achieved through the ketolase gene expression being under the control of a flower-specific promoter. For this purpose, for example, the nucleic acids described above are introduced into the plant, as described in detail below, in a nucleic acid construct functionally linked to a flower-specific promoter.

In a further, particularly preferred embodiment of the process of the invention, genetically modified plants which show the highest rate of expression of a ketolase in fruits are used.

This is preferably achieved through the ketolase gene expression being under the control of a fruit-specific promoter. For this purpose, for example, the nucleic acids described above are introduced into the plant, as described in detail below, in a nucleic acid construct functionally linked to a fruit-specific promoter.

In a further, particularly preferred embodiment of the process of the invention, genetically modified seeds which show the highest rate of expression of a ketolase in seeds are used.

This is preferably achieved through the ketolase gene expression being under the control of a seed-specific promoter. For this purpose, for example, the nucleic acids described above are introduced into the plant, as described in detail below, in a nucleic acid construct functionally linked to a seed-specific promoter.

The targeting into the chromoplasts is effected by a functionally linked plastid transit peptide.

The production of genetically modified plants with increased or caused ketolase activity is described by way of example below. Further activities such as, for example, the hydroxylase activity and/or the β-cyclase activity can be increased analogously using nucleic acid sequences encoding a hydroxylase or β-cyclase in place of nucleic acid sequences encoding a ketolase. The transformation can be effected in the combinations of genetic modifications singly or by multiple constructs.

The transgenic plants are preferably produced by transformation of the starting plants using a nucleic acid construct which comprises the nucleic acids described above encoding a ketolase, which are functionally linked to one or more regulatory signals which ensure transcription and translation in plants.

These nucleic acid constructs in which the coding nucleic acid sequence is functionally linked to one or more regulatory signals which ensure transcription and translation in plants are also called expression cassettes below.

The regulatory signals preferably comprise one or more promoters which ensure transcription and translation in plants.

The expression cassettes comprise regulatory signals, i.e. regulating nucleic acid sequences which control the expression of the coding sequence in the host cell. In a preferred embodiment, an expression cassette comprises a promoter upstream, i.e. at the 5′ end of the coding sequence, and a polyadenylation signal downstream, i.e. at the 3′ end, and, where appropriate, further regulatory elements which are operatively linked to the coding sequence, located in between, for at least one of the genes described above. Operative linkage means the sequential arrangement of promoter, coding sequence, terminator and, where appropriate, further regulatory elements in such a way that each of the regulatory elements is able to carry out its function as intended in the expression of the coding sequence.

The preferred nucleic acid constructs, expression cassettes and vectors for plants and processes for producing transgenic plants, and the transgenic plants themselves, are described by way of example below.

The sequences which are preferred for the operative linkage, but are not restricted thereto, are targeting sequences to ensure the subcellular localization in the apoplast, in the vacuole, in plastids, in the mitochondrion, in the endoplasmic reticulum (ER), in the cell nucleus, in elaioplasts or other compartments and translation enhancers such as the 5′ leader sequence from tobacco mosaic virus (Gallie et al., Nucl. Acids Res. 15 (1987), 8693-8711).

A suitable promoter for the expression cassette is in principle any promoter able to control the expression of foreign genes in plants.

“Constitutive” promoter means promoters which ensure expression in numerous, preferably all, tissues over a relatively wide period during development of the plant, preferably at all times during development of the plant.

Preferably used is, in particular, a plant promoter or a promoter derived from a plant virus. Particular preference is given to the CaMV promoter of the ³⁵S transcript of cauliflower mosaic virus (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228), the 19S CaMV promoter (U.S. Pat. No. 5,352,605; WO 84/02913; Benfey et al. (1989) EMBO J. 8:2195-2202), the triose phosphate translocator (TPT) promoter from Arabidopsis thaliana Acc. No. AB006698, base pair 53242 to 55281; the gene starting at bp 55282 is anotated as “phosphate/triose phosphate translocator”, or the 34S promoter from figwort mosaic virus Acc. No. X16673, base pair 1 to 554.

A further suitable constitutive promoter is the pds promoter (Pecker et al. (1992) Proc. Natl. Acad. Sci USA 89: 4962-4966) or the rubisco small subunit (SSU) promoter (U.S. Pat. No. 4,962,028), the legumin B promoter (GenBank Acc. No. X03677), the agrobacterium nopaline synthase promoter, the TR dual promoter, the agrobacterium OCS (octopine synthase) promoter, the ubiquitin promoter (Holtorf S et al. (1995) Plant Mol Biol 29:637-639), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,633,439), the promoters of the vacuolar ATPase subunits or the promoter of a proline-rich protein from wheat (WO 91/13991), the Pnit promoter (Y07648.L, Hillebrand et al. (1998), Plant. Mol. Biol. 36, 89-99, Hillebrand et al. (1996), Gene, 170, 197-200) and further promoters of genes whose constitutive expression in plants is known to the skilled worker.

The expression cassettes may also comprise a chemically inducible promoter (review article: Gatz et al. (1997) Annu Rev Plant Physiot Plant Mol Biol 48:89-108) by which expression of the ketolase gene in the plant can be controlled at a particular time. Promoters of this type, such as, for example, the PRP1 promoter (Ward et al. (1993) Plant Mol Biol 22:361-366), a salicylic acid-inducible promoter (WO 95/19443), a benzenesulfonamide-inducible promoter (EP 0 388 186), a tetracycline-inducible promoter (Gatz et al. (1992) Plant J 2:397404), an abscisic acid-inducible promoter (EP 0 335 528) or an ethanol- or cyclohexanone-inducible promoter (WO 93/21334), can likewise be used.

Promoters which are further preferred are those induced by biotic or abiotic stress, such as, for example, the pathogen-inducible promoter of the PRP1 gene (Ward et al. (1993) Plant Mol Biol 22:361-366), the heat-inducible tomato hsp70 or hsp80 promoter (U.S. Pat. No. 5,187,267), the cold-inducible potato alpha-amylase promoter (WO 96/12814), the light-inducible PPDK promoter or the wound-induced pinII promoter (EP375091).

Pathogen-inducible promoters include those of genes which are induced as a result of pathogen attack, such as, for example, genes of PR proteins, SAR proteins, β-1,3-glucanase, chitinase etc. (for example Redolfi et al. (1983) Neth J Plant Pathol 89:245-254; Uknes, et al. (1992) The Plant Cell 4:645-656; Van Loon (1985) Plant Mol Viral 4:111-116; Marineau et al. (1987) Plant Mol Biol 9:335-342; Matton et al. (1987) Molecular Plant-Microbe Interactions 2:325-342; Somssich et al. (1986) Proc Natl Acad Sci USA 83:2427-2430; Somssich et al. (1988) Mol Gen Genetics 2:93-98; Chen et al. (1996) Plant J 10:955-966; Zhang and Sing (1994) Proc Natl Acad Sci USA 91:2507-2511; Warner, et al. (1993) Plant J 3:191-201; Siebertz et al. (1989) Plant Cell 1:961-968 (1989).

Also included as wound-inducible promoters such as that of the pinII gene (Ryan (1990) Ann Rev Phytopath 28:425-449; Duan et al. (1996) Nat Biotech 14:494-498), of the wun1 and wun2 genes (U.S. Pat. No. 5,428,148), of the win1 and win2 genes (Stanford et al. (1989) Mol Gen Genet 215:200-208), of the systemin gene (McGurl et al. (1992) Science 255:1570-1573), of the WIP1 gene (Rohmeier et al. (1993) Plant Mol Biol 22:783-792; Ekelkamp et al. (1993) FEBS Letters 323:73-76), of the MPI gene (Corderok et al. (1994) The Plant J 6(2):141-150) and the like.

Examples of further suitable promoters are fruit ripening-specific promoters such as, for example, the tomato fruit ripening-specific promoter (WO 94/21794, EP 409 625). Development-dependent promoters include some of the tissue-specific promoters because the formation of some tissues naturally depends on development.

Further particularly preferred promoters are those which ensure expression in tissues or parts of plant in which, for example, the biosynthesis of ketocarotenoids or precursors thereof takes place. Preferred examples are promoters having specificities for anthers, ovaries, petals, sepals, flowers, leaves, stalks, seeds and roots and combinations thereof.

Examples of promoters specific for tubers, storage roots or roots are the patatin promoter class I (B33) or the potato cathepsin D inhibitor promoter.

Examples of leaf-specific promoters are the promoter of the potato cytosolic FBPase (WO 97/05900), the rubisco (ribulose-1,5-bisphosphate carboxylase) SSU promoter (small subunit) or the potato ST-LSI promoter (Stockhaus et al., (1989) EMBO J. 8:2445-2451).

Examples of flower-specific promoters are the phytoene synthase promoter (WO 92/16635) or the promoter of the P-rr gene (WO 98/22593), the Arabidopsis thaliana AP3 promoter (see example 5), the CHRC promoter (chromoplast-specific carotenoid-associated protein (CHRC) gene promoter from Cucumis sativus Acc. No. AF099501, base pair 1 to 1532), the EPSP synthase promoter (5-enolpyruvylshikimate-3-phosphate synthase gene promoter from Petunia hybrida, Acc. No. M37029, base pair 1 to 1788), the PDS promoter (phytoene desaturase gene promoter from Solanum lycopersicum, Acc. No. U46919, base pair 1 to 2078), the DFR-A promoter (dihydroflavonol 4-reductase gene A promoter from Petunia hybrida, Acc. No. X79723, base pair 32 to 1902) or the FBP1 promoter (floral binding protein 1 gene promoter from Petunia hybrida, Acc. No. L10115, base pair 52 to 1069).

Examples of anther-specific promoters are the 5126 promoter (U.S. Pat. No. 5,689,049, U.S. Pat. No. 5,689,051), the glob-I promoter or the g-zein promoter.

Examples of seed-specific promoters are the ACPO₅ promoter (acyl carrier protein gene, WO 9218634), the Arabidopsis AtS1 and AtS3 promoters (WO 9920775), the Vicia faba LeB4 promoter (WO 9729200 and U.S. Pat. No. 0,640,337,1), the Brassica napus napin promoter (U.S. Pat. No. 5,608,152; EP 255378; U.S. Pat. No. 5,420,034), the Vicia faba SBP promoter (DE 9903432) or the maize End1 and End2 promoters (WO 0011177).

Further promoters suitable for expression in plants are described in Rogers et al. (1987) Meth in Enzymol 153:253-277; Schardl et al. (1987) Gene 61:1-11 and Berger et al. (1989) Proc Natl Acad Sci USA 86:8402-8406.

Particularly preferred in the process of the invention are constitutive, seed-specific, fruit-specific, flower-specific and, in particular, petal-specific promoters.

The present invention therefore relates in particular to a nucleic acid construct comprising functionally linked a flower-specific or, in particular, a petal-specific promoter and a nucleic acid encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

An expression cassette is preferably produced by fusing a suitable promoter to a nucleic acid, described above, encoding a ketolase, and preferably to a nucleic acid which is inserted between promoter and nucleic acid sequence and which codes for a plastid-specific transit peptide, and to a polyadenylation signal by conventional recombination and cloning techniques as described, for example in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1987).

The preferably inserted nucleic acids encoding a plastid transit peptide ensure localization in plastids and, in particular, in chromoplasts.

It is also possible to use expression cassettes whose nucleic acid sequence codes for a ketolase fusion protein, where part of the fusion protein is a transit peptide which controls the translocation of the polypeptide. Transit peptides which are specific for chromoplasts and which are eliminated enzymatically from the ketolase part after translocation of the ketolase into the chromoplasts.

The particularly preferred transit peptide is derived from the Nicotiana tabacum plastid transketolase or another transit peptide (e.g. the transit peptide of the small subunit of rubisco (rbcS) or of the ferredoxin NADP⁺ oxidoreductase, as well as the isopentenyl-pyrophosphate isomerase 2) or its functional equivalent.

Particular preference is given to nucleic acid sequences of three cassettes of the plastid transit peptide of the tobacco plastic transketolase in three reading frames as KpnII/BamHI fragments with an ATG codon in the NcoI cleavage site:

pTP09
Kpnl_GGTACCATGGCGTCTTCTTCTTCTCTCACTCTCTCTCAAGCTATCC
TCTCTCGTTCTGTCCCTCGCCATGGCTCTGCCTCTTCTTCTCAACTTTCC
CCTTCTTCTCTCACTTTTTCCGGCCTTAAATCCAATCCCAATATCACCAC
CTCCCGCCGCCGTACTCCTTCCTCCGCCGCCGCCGCCGCCGTCGTAAGGT
CACCGGCGATTCGTGCCTCAGCTGCAACCGAAACCATAGAGAAAACTGAG
ACTGCGGGATCC_BamHl
pTP10
KPnl_GGTACCATGGCGTCTTCTTCTTCTCTCACTCTCTCTCAAGCTATCC
TCTCTCGTTCTGTCCCTCGCCATGGCTCTGCCTCTTCTTCTCAACTTTCC
CCTTCTTCTCTCACTTTTTCCGGCCTTAAATCCAATCCCAATATCACCAC
CTCCCGCCGCCGTACTCCTTCCTCCGCCGCCGCCGCCGCCGTCGTAAGGT
CACCGGCGATTCGTGCCTCAGCTGCAACCGAAACCATAGAGAAAACTGAG
ACTGCGCTGGATCC_BamHl
pTP11
Kpnl_GGTACCATGGCGTCTTCTTCTTCTCTCACTCTCTCTCAAGCTATCC
TCTCTCGTTCTGTCCCTCGCCATGGCTCTGCCTCTTCTTCTCAACTTTCC
CCTTCTTCTCTCACTTTTTCCGGCCTTAAATCCAATCCCAATATCACCAC
CTCCCGCCGCCGTACTCCTTCCTCCGCCGCCGCCGCCGCCGTCGTAAGGT
CACCGGCGATTCGTGCCTCAGCTGCAACCGAAACCATAGAGAAAACTGAG
ACTGCGGGGATCC_BamHl

Further examples of a plastid transit peptide are the transit peptide of the Arabidopsis thaliana plastid isopentenyl-pyrophosphate isomerase 2 (IPP-2) and the transit peptide of the small subunit of ribulose-bisphosphate carboxylase (rbcS) from pea (Guerineau, F, Woolston, S, Brooks, L, Mullineaux, P (1988) An expression cassette for targeting foreign proteins into the chloroplasts. Nucl. Acids Res. 16:11380).

The nucleic acids of the invention can be prepared synthetically or obtained naturally or comprise a mixture of synthetic and natural nucleic acid constituents, and consist of various heterologous gene sections from different organisms.

Preference is given, as described above, to synthetic nucleotide sequences with codons preferred by plants. These codons preferred by plants can be identified from codons with the highest protein frequency which are expressed in most plant species of interest.

For preparing an expression cassette it is possible to manipulate various DNA fragments in order to obtain a nucleotide sequence which expediently reads in the correct direction and is equipped with a correct reading frame. Adaptors or linkers can be attached to the fragments for connecting the DNA fragments to one another.

It is possible and expedient for the promoter and terminator regions to be provided in the direction of transcription with a linker or polylinker which contains one or more restriction sites for inserting this sequence. As a rule, the linker has 1 to 10, usually 1 to 8, preferably 2 to 6, restriction sites. The linker generally has a size of less than 100 bp, frequently less than 60 bp, but at least 5 bp, inside the regulatory regions. The promoter may be both native or homologous and foreign or heterologous to the host plant. The expression cassette preferably comprises in the 5′-3′ direction of transcription the promoter, a coding nucleic acid sequence or a nucleic acid construct and a region for termination of transcription. Various termination regions are interchangeable as desired.

Examples of a terminator are the 35S terminator (Guerineau et al. (1988) Nuci Acids Res. 16: 11380), the nos terminator (Depicker A, Stachel S, Dhaese P, Zambryski P, Goodman H M. Nopaline synthase: transcript mapping and DNA sequence. J Mol Appl Genet. 1982; 1(6):561-73) or the ocs terminator (Gielen, J, de Beuckeleer, M, Seurinck, J, Debroek, H, de Greve, H, Lemmers, M, van Montagu, M, Schell, J (1984) The complete sequence of the TL-DNA of the Agrobacterium tumefaciens plasmid pTiAch5. EMBO J. 3: 835-846).

It is furthermore possible to employ manipulations which provide appropriate restriction cleavage sites or delete the redundant DNA or restriction cleavage sites. It is possible in relation to insertions, deletions or substitutions, such as, for example, transitions and transversions, to use in vitro mutagenesis, primer repair, restriction or ligation.

It is possible with suitable manipulations, such as, for example, restriction, chewing back or filling in of overhangs for blunt ends, to provide complementary ends of the fragments for ligation.

Preferred polyadenylation signals are plant polyadenylation signals, preferably those which essentially correspond to T-DNA polyadenylation signals from Agrobacterium tumefaciens, especially of gene 3 of the T-DNA (octopine synthase) of the Ti plasmid pTiACH5 (Gielen et al., EMBO J. 3 (1984), 835 ff) or functional equivalents.

The transfer of foreign genes into the genome of a plant is referred to as transformation.

It is possible to use for this purpose methods known per se for the transformation and regeneration of plants from plant tissues or plant cells for transient or stable transformation.

Suitable methods for transforming plants are protoplast transformation by polyethylene glycol-induced DNA uptake, the biolistic method using the gene gun—called the particle bombardment method—electroporation, incubation of dry embryos in DNA-containing solution, microinjection and gene transfer mediated by Agrobacterium described above. Said processes are described, for example, in B. Jenes et al., Techniques for Gene Transfer, in: Transgenic Plants, Vol. 1, Engineering and Utilization, edited by S. D. Kung and R. Wu, Academic Press (1993), 128-143 and in Potrykus, Annu. Rev. Plant Physiol. Plant Molec. Biol. 42 (1991), 205-225.

The construct to be expressed is preferably cloned into a vector which is suitable for transforming Agrobacterium tumefaciens, for example pBin19 (Bevan et al., Nucl. Acids Res. 12 (1984), 8711) or particularly preferably, pSUN2, pSUN3, pSUN4 or pSUN5 (WO 02/00900).

Agrobacteria transformed with an expression plasmid can be used in a known manner for transforming plants, e.g. bathing wounded leaves or pieces of leaf in a solution of agrobacteria and subsequently cultivating in suitable media.

For the preferred production of genetically modified plants, also referred to as transgenic plants hereinafter, the fused expression cassette which expresses a ketolase is cloned into a vector, for example pBin19 or, in particular, pSUN5 and pSUN3, which is suitable for being transformed into Agrobacterium tumefaciens. Agrobacteria transformed with such a vector can then be used in a known manner for transforming plants, in particular crop plants, by bathing wounded leaves or pieces of leaf in a solution of agrobacteria and subsequently cultivating in suitable media.

The transformation of plants by agrobacteria is disclosed inter alia in F. F. White, Vectors for Gene Transfer in Higher Plants; in Transgenic Plants, Vol. 1, Engineering and Utilization, edited by S. D. Kung and R. Wu, Academic Press, 1993, pages 15-38. Transgenic plants which comprise a gene, integrated into the expression cassette for expression of a nucleic acid encoding a ketolase can be regenerated in a known manner from the transformed cells of the wounded leaves or pieces of leaf.

To transform a host cell with a nucleic acid coding for a ketolase, an expression cassette is incorporated and inserted into a recombinant vector whose vector DNA comprises additional functional regulatory signals, for example sequences for replication or integration. Suitable vectors are described inter alia in “Methods in Plant Molecular Biology and Biotechnology” (CRC Press), chapter 6/7, pages 71-119 (1993).

Using the recombination and cloning techniques quoted above, the expression cassettes can be cloned into suitable vectors which make replication thereof possible for example in E. coli. Suitable cloning vectors are, inter alia, pJIT117 (Guerineau et al. (1988) Nucl. Acids Res. 16:11380), pBR322, pUC series, M13 mp series and pACYC184. Binary vectors which are able to replicate both in E. coli and in agrobacteria are particularly suitable.

The production of the genetically modified microorganisms of the invention is described in more detail below:

The nucleic acids described above, encoding a ketolase or hydroxylase or β-cyclase, are preferably incorporated into expression constructs comprising, under the genetic control of regulatory nucleic acid sequences, a nucleic acid sequence coding for an enzyme of the invention; and vectors comprising at least one of these expression constructs.

Such constructs of the invention preferably include a promoter upstream, i.e. at the 5′ end of the particular coding sequence, and a terminator sequence downstream, i.e. at the 3′ end, and, where appropriate, further customary regulatory elements which are in each case operatively linked to the coding sequence. Operative linkage means the sequential arrangement of promoter, coding sequence, terminator and, where appropriate, further regulatory elements in such a way that each of the regulatory elements is able to carry out its function as intended in the expression of the coding sequence.

Examples of operatively linkable sequences are targeting sequences and translation enhancers, polyadenylation signals and the lilke. Further regulatory elements include selectable markers, amplification signals, origins of replication and the like.

In addition to the artificial regulatory sequences it is possible for the natural regulatory sequence still to be present in front of the actual structural gene. This natural regulation can be switched off where appropriate, and the expression of the genes increased or reduced, by genetic modification. The gene construct may, however, also have a simpler structure, that is to say no additional regulatory signals are inserted in front of the structural gene, and the natural promoter with its regulation is not deleted. Instead, the natural regulatory sequence is mutated so that regulation no longer takes place, and gene expression is increased or reduced. The nucleic acid sequences may be present in one or more copies in the gene construct.

Examples of promoters which can be used are: cos, tac, trp, tet, trp-tet, lpp, lac, lpp-lac, laclq, T7, T5, T3, gal, trc, ara, SP6, lambda-PR or lambda-PL promoter, which are advantageously used in Gram-negative bacteria; and the Gram-positive promoters amy and SPO2 or the yeast promoters ADC1, MFα, AC, P-60, CYC1, GAPDH. The use of inducible promoters is particularly preferred, such as, for example, light- and, in particular, temperature-inducible promoters such as the PᵣPₗ promoter.

It is possible in principle for all natural promoters with their regulatory sequences to be used. In addition, it is also possible advantageously to use synthetic promoters.

Said regulatory sequences are intended to make specific expression of the nucleic acid sequences and protein expression possible. This may mean, for example, depending on the host organism, that the gene is expressed or overexpressed only after induction or that it is immediately expressed and/or overexpressed.

The regulatory sequences or factors may moreover preferably influence positively, and thus increase or reduce, expression. Thus, enhancement of the regulatory elements can take place advantageously at the level of transcription by using strong transcription signals such as promoters and/or enhancers. However, it is also possible to enhance translation by, for example, improving the stability of the mRNA.

An expression cassette is produced by fusing a suitable promoter to the above described nucleic acid sequence which encodes a ketolase, β-hydroxylase or β-cyclase and to a terminator signal or polyadenylation signal. Conventional techniques of recombination and cloning are used for this purpose, as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley Interscience (1987).

For expression in a suitable host organism, the recombinant nucleic acid construct or gene construct is advantageously inserted into a host-specific vector, which makes optimal expression of the genes in the host possible. Vectors are well known to the skilled worker and can be found, for example, in “Cloning Vectors” (Pouwels P. H. et al., eds, Elsevier, Amsterdam-New York-Oxford, 1985). Vectors also mean not only plasmids but also all other vectors known to the skilled worker, such as, for example, phages, viruses, such as SV40, CMV, baculovirus and adenovirus, transposons, IS elements, phasmids, cosmids, and linear or circular DNA. These vectors may undergo autonomous replication in the host organism or chromosomal replication.

Examples of suitable expression vectors which may be mentioned are:

Conventional fusion expression vectors such as pGEX (Pharmacia Biotech Inc; Smith, D. B. and Johnson, K. S. (1988) Gene 67:31-40), pMAL (New England Biolabs, Beverly, Mass.) and pRIT 5 (Pharmacia, Piscataway, N.J.), with which respectively glutathione S-transferase (GST), maltose E-binding protein and protein A are fused to the recombinant target protein.

Non-fusion protein expression vectors such as pTrc (Amann et al., (1988) Gene 69:301-315) and pET 11d (Studier et al. Gene Expression Technology: Methods in Enzymology 185, Academic Press, San Diego, Calif. (1990) 60-89).

Yeast expression vector for expression in the yeast S. cerevisiae, such as pYepSec1 (Baldari et al., (1987) Embo J. 6:229-234), pMFα (Kurjan and Herskowitz (1982) Cell 30:933-943), pJRY88 (Schultz et al. (1987) Gene 54:113-123) and pYES2 (Invitrogen Corporation, San Diego, Calif.).

Vectors and methods for constructing vectors suitable for the use in other fungi such as filamentous fungi comprise those which are described in detail in: van den Hondel, C. A. M. J. J. & Punt, P. J. (1991) “Gene transfer systems and vector development for filamentous fungi, in: Applied Molecular Genetics of Fungi, J. F. Peberdy et al., eds, pp. 1-28, Cambridge University Press: Cambridge.

Baculovirus vectors which are available for expression of proteins in cultured insect cells (for example Sf9 cells) comprise the pAc series (Smith et al., (1983) Mol. Cell Biol. 3:2156-2165) and pVL series (Lucklow and Summers (1989) Virology 170:31-39).

Further suitable expression systems for prokaryotic and eukaryotic cells are described in chapters 16 and 17 of Sambrook, J., Fritsch, E. F. and Maniatis, T., Molecular cloning: A Laboratory Manual, 2nd edition, Cold Spring Harbor Laboratory, Cold Spring Harbor Laboratory Press, Cold Spring Harbor, N.Y., 1989.

The expression constructs or vectors of the invention can be used to produce genetically modified microorganisms which are transformed, for example, with at least one vector of the invention.

The recombinant constructs of the invention described above are advantageously introduced and expressed in a suitable host system. Cloning and transfection methods familiar to the skilled worker, such as, for example, coprecipitation, protoplast fusion, electroporation, retroviral transfection and the like, are preferably used to bring about expression of said nucleic acids in the particular expression system. Suitable systems are described, for example, in Current Protocols in Molecular Biology, F. Ausubel et al., eds, Wiley Interscience, New York 1997.

Successfully transformed organisms can be selected through marker genes which are likewise present in the vector or in the expression cassette. Examples of such marker genes are genes for antibiotic resistance and for enzymes which catalyze a color-forming reaction which causes staining of the transformed cell. These can then be selected by automatic cell sorting.

Microorganisms which have been successfully transformed with a vector and harbor an appropriate antibiotic resistance gene (for example G418 or hygromycin) can be selected by appropriate antibiotic-containing media or nutrient media. Marker proteins present on the surface of the cell can be used for selection by means of affinity chromatography.

The combination of the host organisms and the vectors appropriate for the organisms, such as plasmids, viruses or phages, such as, for example, plasmids with the RNA polymerase/promoter system, phages 8 or other temperate phages or transposons and/or other advantageous regulatory sequences forms an expression system.

The invention further relates to a process for producing genetically modified organisms, which comprises introducing a nucleic acid construct comprising functionally linked a promoter and nucleic acids encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, and, where appropriate, a terminator into the genome of the starting organism or extrachromosomally into the starting organism.

The invention further relates to the genetically modified organisms where the genetic modification
A in the case where the wild-type organism already has a ketolase activity, increases the activity of a ketolase compared with the wild type and
B in the case where the wild-type organism has no ketolase activity, causes the activity of a ketolase compared with the wild type,
   and the ketolase activity which has been increased as in A or caused as in B is caused by a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

As stated above, the increasing or causing of the ketolase activity is brought about by an increasing or causing of the gene expression of a nucleic acid encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2, compared with the wild type.

In a further preferred embodiment, as stated above, the increasing or causing of the gene expression of a nucleic acid encoding a ketolase takes place by introducing nucleic acids encoding a ketolase into the plants and thus preferably by overexpression or transgenic expression of nucleic acids encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2.

The invention further relates to a genetically modified organism comprising at least one transgenic nucleic acid encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2. This is the case when the starting organism has no ketolase or an endogenous ketolase, and a transgenic ketolase is overexpressed.

The invention further relates to a genetically modified organism comprising at least two endogenous nucleic acids encoding a ketolase comprising the amino acid sequence SEQ. ID. NO. 2 or a sequence which is derived from this sequence by substitution, insertion or deletion of amino acids and which has an identity of at least 42% at the amino acid level with the sequence SEQ. ID. NO. 2. This is the case when the starting organism has an endogenous ketolase, and the endogenous ketolase is overexpressed.

Particularly preferred genetically modified organisms have, as mentioned above, additionally an increased hydroxylase activity and/or β-cyclase activity compared with a wild-type organism. Further preferred embodiments are described above in the process of the invention.

Organisms preferably mean according to the invention organisms which are able as wild-type or starting organisms naturally or through genetic complementation and/or reregulation of metabolic pathways to produce carotenoids, in particular β-carotene and/or zeaxanthin and/or neoxanthin and/or violaxanthin and/or luteine.

Further preferred organisms already have as wild-type or starting organisms a hydroxylase activity and are thus able as wild-type or starting organisms to produce zeaxanthin.

Preferred organisms are plants or microorganisms such as, for example, bacteria, yeasts, algae or fungi.

Bacteria which can be used are both bacteria which are able, because of the introduction of genes of carotenoid biosynthesis of a carotenoid-producing organism, to synthesize xanthophylls, such as, for example, bacteria of the genus Escherichia, which comprise for example crt genes from Erwinia, and bacteria which are intrinsically able to synthesize xanthophylls, such as, for example, bacteria of the genus Erwinia, Agrobacterium, Flavobacterium, Alcaligenes, Paracoccus, Nostoc or cyanobacteria of the genus Synechocystis.

Preferred bacteria are Escherichia coli, Erwinia herbicola, Erwinia uredovora, Agrobacterium aurantiacum, Alcaligenes sp. PC-1, Flavobactenum sp. strain R1534, the cyanobacterium Synechocystis sp. PCC6803, Paracoccus marcusii or Paracoccus carotinifaciens.

Preferred yeasts are Candida, Saccharomyces, Hansenula, Pichia or Phaffia. Particularly preferred yeasts are Xanthophyllomyces dendrorhous or Phaffia rhodozyma.

Preferred fungi are Aspergillus, Trichoderma, Ashbya, Neurospora, Blakeslea, Phycomyces, Fusarium or other fungi described in Indian Chem. Engr. Section B. Vol. 37, No. 1, 2 (1995) on page 15, table 6.

Preferred algae are green algae such as, for example, algae of the genus Haematococcus, Phaedactylum tricomatum, Volvox or Dunaliella. Particularly preferred algae are Haematococcus pluvialis or Dunaliella bardawil.

Further microorganisms which can be used and the production thereof for carrying out the process of the invention are disclosed for example in DE-A-199 16 140, which is incorporated herein by reference.

Particularly preferred plants are plants selected from the families Ranunculaceae, Berberidaceae, Papaveraceae, Cannabaceae, Rosaceae, Fabaceae, Linaceae, Vitaceae, Brassicaceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, llliaceae or Lamiaceae.

Very particularly preferred plants are selected from the group of plant genera Marigold, Tagetes errecta, Tagetes patula, Acacia, Aconitum, Adonis, Amica, Aquilegia, Aster, Astragalus, Bignonia, Calendula, Caftha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hibiscus, Heliopsis, Hypencum, Hypochoeris, Impatiens, Iris, Jacaranda, Kerria, Labumum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola or Zinnia, particularly preferably selected from the group of plant genera Marigold, Tagetes erecta, Tagetes patula, Lycopersicon, Rosa, Calendula, Physalis, Medicago, Helianthus, Chrysanthemum, Aster, Tulipa, Narcissus, Petunia, Geranium, Tropaeolum or Adonis.

Very particularly preferred genetically modified plants are selected from the plant genera Marigold, Tagetes erecta, Tagetes patula, Adonis, Lycopersicon, Rosa, Calendula, Physalis, Medicago, Helianthus, Chrysanthemum, Aster, Tulipa, Narcissus, Petunia, Geranium or Tropaeolum, with the genetically modified plant comprising at least one transgenic nucleic acid encoding a ketolase.

The present invention further relates to the transgenic plants, their propagation material, and their plant cells, tissues or parts, especially their fruit, seeds, flowers and petals.

The genetically modified plants can, as described above, be used for preparing ketocarotenoids, especially astaxanthin.

Genetically modified organisms of the invention which can be consumed by humans and animals, especially plants or parts of plants, such as, in particular, petals with an increased content of ketocarotenoids, especially astaxanthin, can also be used directly or after processing known per se as human or animal foods or as animal and human food supplements.

The genetically modified organisms can also be used for producing ketocarotenoid-containing extracts of the organisms and/or for producing animal and human food supplements.

The genetically modified organisms have an increased content of ketocarotenoids compared with the wild type.

An increased content of ketocarotenoids usually means an increased total ketocarotenoid content.

However, an increased content of ketocarotenoid also means in particular an altered content of the preferred ketocarotenoids without the need for the total carotenoid content necessarily to be increased.

In a particularly preferred embodiment, the genetically modified plants of the invention have an increased astaxanthin content compared with the wild type.

An increased content means in this case also a caused content of ketocarotenoids such as astaxanthin.

The invention further relates to the novel ketolases and to the novel nucleic acids which encode the latter.

The invention relates in particular to ketolases comprising the amino acid sequence SEQ. ID. NO. 2 or a sequence which is derived from this sequence by substitution, insertion or deletion of amino acids and which has an identity of at least 70%, preferably at least 75%, particularly preferably at least 80%, more preferably at least 85%, more preferably at least 90%, more preferably at least 95% at the amino acid level with the sequence SEQ. ID. NO. 2, with the proviso that the amino acid sequence SEQ. ID NO. 2 is not present. The sequence SEQ ID NO: 2 is, as mentioned above, annotated as putative protein in databases.

The invention further relates to ketolases comprising the amino acid sequence SEQ. ID. NO. 4 or a sequence which is derived from this sequence by substitution, insertion or deletion of amino acids and which has an identity of at least 70% at the amino acid level with the sequence SEQ. ID. NO. 4. The sequence SEQ ID NO: 4 is, as mentioned above, not annotated in databases.

The invention further relates to nucleic acids encoding a protein described above, with the proviso that the nucleic acid does not comprise the sequences SEQ ID NO: 1 or 3.

It has surprisingly been found that a protein comprising the amino acid sequence SEQ. ID. NO. 2 or a sequence which is derived from this sequence by substitution, insertion or deletion of amino acids and which has an identity of at least 70%, preferably at least 75%, particularly preferably at least 80%, more preferably at least 85%, more preferably at least 90%, more preferably at least 95%, at the amino acid level with the sequence SEQ. ID. NO. 2 and has the property of a ketolase, has a property as ketolase.

The invention therefore also relates to the use of a protein comprising the amino acid sequence SEQ. ID. NO. 2 or a sequence which is derived from this sequence by substitution, insertion or deletion of amino acids and which has an identity of at least 70%, preferably at least 75%, particularly preferably at least 80%, more preferably at least 85%, more preferably at least 90%, more preferably at least 95%, at the amino acid level with the sequence SEQ. ID. NO. 2, and has the property of a ketolase, as ketolase.

It has also surprisingly been found that a protein comprising the amino acid sequence SEQ. ID. NO. 4 or a sequence which is derived from this sequence by substitution, insertion or deletion of amino acids and which has an identity of at least 65%, preferably at least 70%, preferably at least 75%, particularly preferably at least 80%, more preferably at least 85%, more preferably at least 90%, more preferably at least 95%, at the amino acid level with the sequence SEQ. ID. NO. 4, and has the property of a ketolase, has a property as ketolase.

The invention therefore also relates to the use of a protein comprising the amino acid sequence SEQ. ID. NO. 4 or a sequence which is derived from this sequence by substitution, insertion or deletion of amino acids and which has an identity of at least 65%, preferably at least 70%, preferably at least 75%, particularly preferably at least 80%, more preferably at least 85%, more preferably at least 90%, more preferably at least 95%, at the amino acid level with the sequence SEQ. ID. NO. 4, and has the property of a ketolase, as ketolase.

Compared with prior art processes, the process of the invention affords a larger quantity of ketocarotenoids, especially astaxanthin.

The invention is now explained by the following examples, but is not restricted thereto:

### EXAMPLE 1

Amplification of cDNA, which encodes a complete primary sequence of the ketolases from Nostocpunctiforme PCC73102 ORF 38, contig 501 (SEQ ID NO: 1) and ORF 148, contig 502 (SEQ ID NO: 3)

Cells of Nostoc punctiforme were disrupted with lysozyme (2 mg/ml) and the genomic DNA was isolated with the aid of the GenElute Plant genomic DNA kit (Sigma) in accordance with the manufacturer's information.

This was followed by amplification of ORF148 (762 bp) from the genomic DNA of Nostoc punctiforme with the aid of the primers 148-Start (SEQ ID NO: 9; 5′ ATG ATC CAG TTA GM CAA CCA C-3′) and 148-End (SEQ ID NO: 10; 5′CTA TTT TGC TTT GTA AAT TTC TGG-3′) at an annealing temperature of 60° C. over 30 cycles.

ORF38 (789 bp) was amplified using the primers 38-Start (SEQ ID NO: 11; 5′ ATG AAT TTT TGT GAT MA CCA GTT AG-3′) and 38-End (SEQ ID NO: 12; 5′ ACG MT TGG TTA CTG MT TGT TG-3′).

The PCR fragments were subcloned into the Xcml-cut vector pMON 38201 (Borokov, A. Y. and Rivkin, M. I. (1997) Xcml containing vector for direct cloning of pcr products. BioTech. 22, 812-814).

Positive clones were selected by carrying out a blue-white screening after transformation of the ligation products into XL1 blue MRF1′. The isolated plasmid DNA was cut with HindIII in order to check whether the PCR amplicon was cloned into the T overhang vector. Sequencing of the selected clones showed that the orientation of ORF148 in pMONT-148, and of ORF38 in pMONT-38, is contrary to the vectorial reading direction. It was possible to cut out the insert with HindIII because the T overhang vector possesses not only the HindIII cleavage site in the polylinker but also a second one produced on insertion of the polylinker.

### EXAMPLE 2

Preparation of expression vectors for expression of the Nostoc punctiforme PCC73102 ketolases ORF148 and ORF38 in host organisms.

After restriction digestion of pMONT148 and pMONT38 with HindIII, the resulting DNA inserts were cloned into a pPQE32 vector (Qiagen, Hilden; modified as described in Verdoes, J., Krubasik, P., Sandmann, G. & van Ooyen, M. (1999) Isolation and functional characterisation of a novel type of carotenoid biosynthetic gene from Xanthophyllomyces dendrorhous. Molec. Gen. Genet. 262, 453-461 which had likewise been digested with HindIII and dephosphorylated.

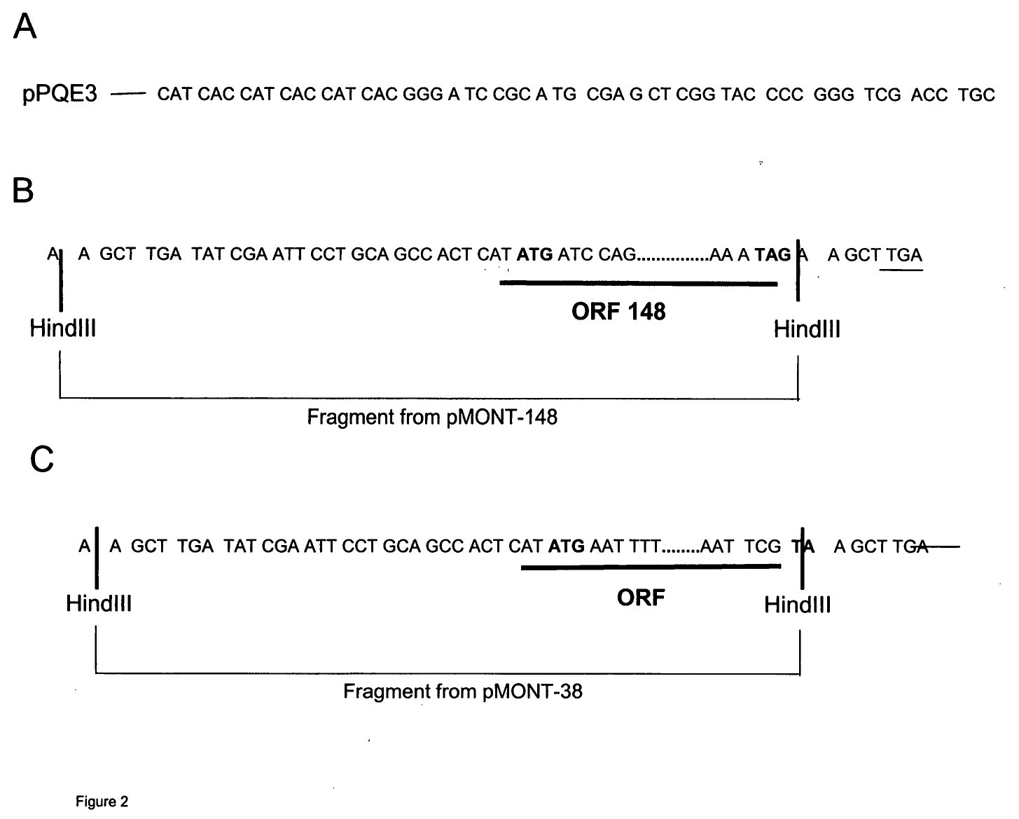

The clones obtained after transformation into XL1MRF1′ were examined by means of a check PCR using primer QEF (5′CCC TTT CCT CTT CTC-3′) and 148-end or 38-end. The sequencings of the corresponding clones showed that ORF148 and ORF38 were cloned in frame ito the pPQE32 vector. The plasmids obtained in this way are depicted in FIGS. 2B and 2C. FIG. 2 shows the construction of pPQE32-ORF 148 (B.) and pPQE32-ORF 38 (C.) starting from pPQE32 (A.).

### EXAMPLE 3

Expression of the Nostoc punctiforme PCC73102 ketolases ORF148 and ORF38 in β-carotene and zeaxanthin producing E. coli strains and analysis of the carotenoid profile

3.1. Expression of the Nostoc punctiforme PCC73102 ketolases ORF148 and ORF38 in β-carotene producing E. coli strains

For functional characterization of the gene products formed by ORF148 and ORF38, the constructs pPQE32-148 and pPQE32-38 were transformed into the β-carotene producing E. coli transformant JM101/pACCAR16ΔcrtX (Misawa, N., Satomi, Y., Kondo, K., Yokoyama, A., Kajiwara, S., Saito, T. Ohtani, T. & Miki, W. (1995) Structure and functional analysis of a marine bacterial carotenoid biosynthesis gene cluster and astaxanthin biosynthetic pathway proposed at the gene level. J. Bacteriol. 22, 6575-6584).

The transformants were cultured in 50 ml cultures with LB medium at 28° C. in the dark for 16 to 48 hours. The carotenoids were extracted with methanol, and the extracts obtained by shaking with 50% ether/petroleum ether were fractionated by HPLC (column HypurityC18, mobile phase: acetonitrile/methanol/2-propanol 85:10:5, temperature 32° C.). The spectra were recorded on-line by means of a diode array detector, and the carotenoids were identified on the basis of their absorption maxima and by comparison with standards.

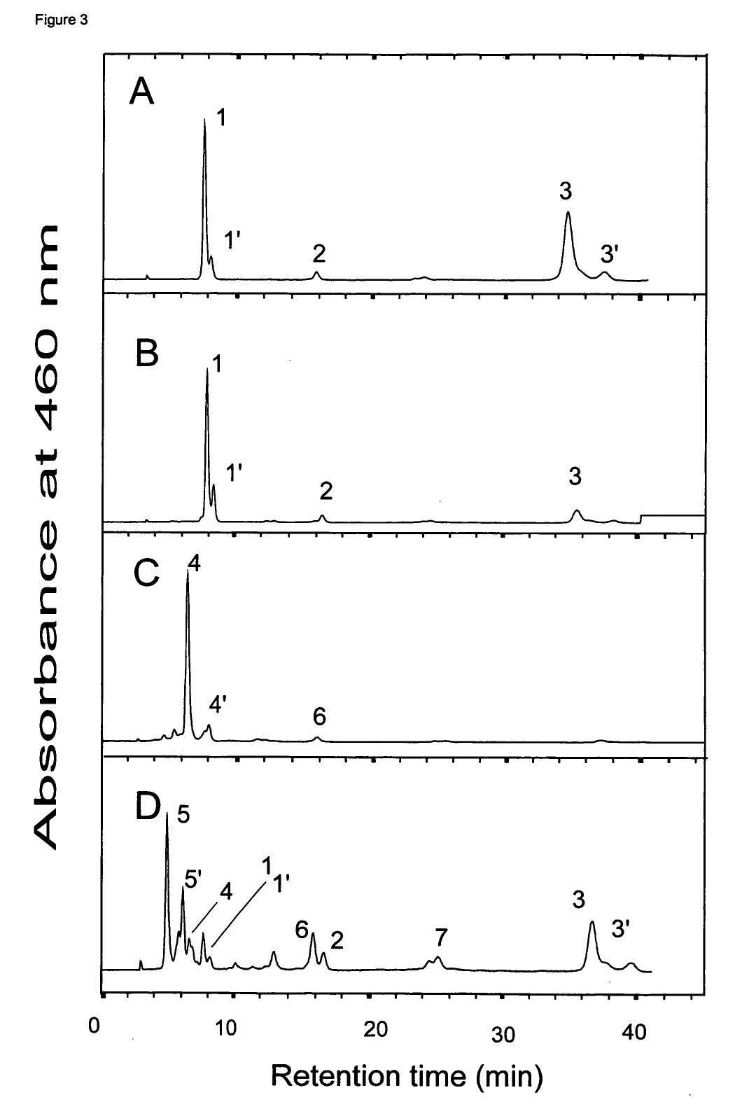

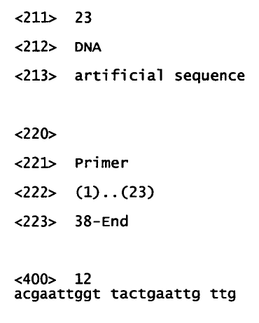

As shown in FIG. 3A for pPQE32-38 and 3B for pPQE32-148, it was possible to detect, besides an initial substrate β-carotene, in both extracts the ketocarotenoids echinenone and canthaxanthin (in controls without pPQE32-38 or pPQE32-148, only β-carotene but no ketocarotenoids was to be found).

The proportion of canthaxarithin (diketo compound) produced in the total carotenoid content was 81% on complementation with pPQE32-148 and 40% on complementation with pPQE32-38. The proportion of echinenone (monoketo compound) was about 4% with both complementations.

3.2. Expression of the Nostoc punctiforme PCC73102 ketolases ORF148 and ORF38 in zeaxanthin producing E. coli strains

In order to investigate how far the ketolases encoded by ORF148 and ORF38 are able to synthesize the ketocarotenoid astaxanthin, pPQE32-38 (FIG. 3C) and pPQE32-148 (FIG. 3D) were transformed into the zeaxanthin producing E. coli transformants JM101/pACCAR25ΔcrtX (Misawa, N., Satomi, Y., Kondo, K., Yokoyama, A., Kajiwara, S., Saito, T. Ohtani, T. & Miki, W. (1995) Structure and functional analysis of a marine bacterial carotenoid biosynthesis gene cluster and astaxanthin biosynthetic pathway proposed at the gene level. J. Bacteriol. 22, 6575-6584).

The culturing of the transformants, the carotenoid extraction and the HPLC separation took place as described above in 3.1. Whereas only the initial substrates zeaxanthin and β-carotene, respectively 85 and 5% of the total carotenoid content, were detectable in the extract obtained from complementation with pPQE32-38, chiefly the ketocarotenoids echinenone, canthaxanthin and astaxanthin were detectable on complementation with pPQE32-148. The proportion of astaxanthin in the total carotenoid content was 50%. The intermediates of astaxanthin synthesis, echinenone and canthaxanthin, represent respectively 12% and 8% of the total carotenoid. The proportion of β-carotene is about 30%.

FIG. 3 shows the HPLC separation of the carotenoids from complementation in E. coli with a β-carotene background cotransformed with pPQE32-38 (A) or pPQE32-148 (B) and in E. coli with a zeaxanthin background cotransformed with pPQE32-38 (C) or pPQE32-148 (D).

The stated carotenoids were identified by cochromatography with comparison substances and via their spectra as:
1 Canthaxanthin,
2 Echinenone,
3 β-Carotene,
4 Zeaxanthin,
5 astaxanthin,
6 β-Cryptoxanthin,
7 Neurosporin.

1′, 3′, 4′ and 5′ designate the corresponding cis isomers.

1. A process for preparing ketocarotenoids by cultivating genetically modified organisms which, compared with the wild type, have a modified ketolase activity, and the modified ketolase activity is caused by a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2.

2. The process as claimed in claim 1, wherein organisms which, as wild type, already have a ketolase activity, and the genetic modification brings about an increase in the ketolase activity compared with the wild type, are used.

3. The process as claimed in claim 1, wherein the ketolase activity is increased by increasing the gene expression of a nucleic acid encoding a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, compared with the wild type.

4. The process as claimed in claim 3, wherein the gene expression is increased by introducing nucleic acids which encode ketolases comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, compared with the wild type, into the organism.

5. The process as claimed in claim 1, wherein organisms which, as wild type, have no ketolase activity are used, and the genetic modification causes a ketolase activity compared with the wild type.

6. The process as claimed in claim 5, wherein genetically modified organisms which transgenically express a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, are used.

7. The process as claimed in claim 5, wherein the gene expression is caused by introducing nucleic acids which encode ketolases comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, into the organism

8. The process as claimed in claim 5, wherein nucleic acids comprising the sequence SEQ ID NO:1 are introduced.

9. The process as claimed in claim 1, wherein the organisms additionally have an increased activity, compared with the wild type, of at least one of the activities selected from the group consisting of hydroxylase activity and β-cyclase activity.

10. The process as claimed in claim 9, wherein the gene expression of at least one nucleic acid selected from the group consisting of nucleic acids encoding a hydroxylase, and nucleic acids encoding a β-cyclase, is increased compared with the wild type for the additional increase in at least one of the activities.

11. The process as claimed in claim 10, wherein the gene expression is increased by introducing at least one nucleic acid selected from the group consisting of nucleic acids encoding a hydroxylase and nucleic acids encoding a β-cyclase into the organism.

12. The process as claimed in claim 11, wherein nucleic acids which encode a hydroxylase comprising the amino acid sequence SEQ ID NO: 6 or a sequence which is derived from this sequence by substitution, insertion or deletion of amino acids and which has an identity of at least 20% at the amino acid level with the sequence SEQ ID NO: 6 are introduced as nucleic acid encoding a hydroxylase.

13. The process as claimed in claim 12, wherein nucleic acids comprising the sequence SEQ ID NO: 5 are introduced.

14. The process as claimed in claim 11, wherein nucleic acids which encode a β-cyclase comprising the amino acid sequence SEQ ID NO: 8 or a sequence which is derived from this sequence by substitution, insertion or deletion of amino acids and which has an identity of at least 20% at the amino acid level with the sequence SEQ ID NO: 8 are introduced as nucleic acid encoding a β-cyclase.

15. The process as claimed in claim 14, wherein nucleic acids comprising the sequence SEQ ID NO: 7 are introduced.

16. The process as claimed in claim 1, wherein the genetically modified organisms are harvested after cultivation, and subsequently the ketocarotenoids are isolated from the organisms.

17. The process as claimed in claim 1, wherein an organism which is able as starting organism naturally or through genetic complementation or reregulation of metabolic pathways to produce carotenoids is used as organism.

18. The process as claimed in claim 1, wherein microorganisms or plants are used as organisms.

19. The process as claimed in claim 18, wherein bacteria, yeasts, algae or fungi are used as microorganisms.

20. The process as claimed in claim 19, wherein the microorganisms are selected from the group consisting of Escherichia, Erwinia, Agrobacterium, Flavobacterium, Alcaligenes, Paracoccus, Nostoc, cyanobacteria of the genus Synechocystis, Candida, Saccharomyces, Hansenula, Phaffia, Pichia, Aspergillus, Trichoderma, Ashbya, Neurospora, Blakeslea, Phycomyces, Fusarium, Haematococcus, Phaedactylum tricornatum, Volvox and Dunaliella.

21. The process as claimed in claim 18, wherein plants are used as organism.

22. The process as claimed in claim 21, wherein a plant selected from the families consisting of Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae and Lamiaceae is used as plant.

23. The process as claimed in claim 22, wherein a plant selected from the plant genera consisting of Marigold, Tagetes erecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hibiscus, Heliopsis, Hypericum, Hypochoeris, Impatiens, Iris, Jacaranda, Kerria, Laburnum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola and Zinnia is used as plant.

24. The process as claimed in claim 1, wherein the ketocarotenoids are selected from the group consisting of astaxanthin, canthaxanthin, echinenone, 3-hydroxyechinenone, 3′-hydroxyechinenone, adonirubin and adonixanthin.

25. A genetically modified organism where the genetic modification
A, in the case where the wild-type organism already has a ketolase activity, increases the activity of a ketolase compared with the wild type and
B, in the case where the wild-type organism has no ketolase activity, causes the activity of a ketolase compared with the wild type,
and the ketolase activity which has been increased as in A or caused as in B is caused by a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2.

26. The genetically modified organism as claimed in claim 25, wherein the increasing or causing of the ketolase activity is brought about by an increasing or causing of the gene expression of a nucleic acid encoding a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, compared with the wild type.

27. The genetically modified organism as claimed in claim 26, wherein to increase or cause the gene expression nucleic acids which encode ketolases comprising the amino acid sequence SEQ ID NO: 2 or a sequence which is derived from this sequence by substitution, insertion or deletion of amino acids and which has an identity of at least 42% at the amino acid level with the sequence SEQ ID NO: 2, are introduced into the organism.

28. A genetically modified organism comprising at least one transgenic nucleic acid encoding a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2.

29. A genetically modified organism comprising at least two endogenous nucleic acids encoding a ketolase comprising the amino acid sequence SEQ ID NO: 2 or a sequence which is derived from this sequence by substitution, insertion or deletion of amino acids and which has an identity of at least 42% at the amino acid level with the sequence SEQ ID NO: 2.

30. The genetically modified organism as claimed in claim 25, wherein the genetic modification additionally increases at least one of the activities selected from the group consisting of hydroxylase activity and β-cyclase activity, compared with the wild type.

31. The genetically modified organism as claimed in claim 25, which is able as starting organism naturally or through genetic complementation to produce carotenoids.

32. The genetically modified organism as claimed in claim 25, selected from the group consisting of microorganisms OF and plants.

33. The genetically modified organism as claimed in claim 32, wherein the microorganisms are selected from the group consisting of bacteria, yeasts, algae of and fungi.

34. The genetically modified microorganism as claimed in claim 33, wherein the microorganisms are selected from the group consisting of Escherichia, Erwinia, Agrobacterium, Flavobacterium, Alcaligenes, Paracoccus, Nostoc, cyanobacteria of the genus Synechocystis, Candida, Saccharomyces, Hansenula, Pichia, Aspergillus, Trichoderma, Ashbya, Neurospora, Blakeslea, Phycomyces, Fusarium, Haematococcus, Phaedactylum tricornatum, Volvox and Dunaliella.

35. The genetically modified plant as claimed in claim 32, wherein the plants are selected from the families consisting of Ranunculaceae, Berberidaceae, Papaveraceae, Cannabaceae, Rosaceae, Fabaceae, Linaceae, Vitaceae, Brassiceae, Cucurbitaceae, Primulaceae, Caryophyllaceae, Amaranthaceae, Gentianaceae, Geraniaceae, Caprifoliaceae, Oleaceae, Tropaeolaceae, Solanaceae, Scrophulariaceae, Asteraceae, Liliaceae, Amaryllidaceae, Poaceae, Orchidaceae, Malvaceae, Illiaceae and Lamiaceae.

36. The genetically modified plant as claimed in claim 35, wherein the plants are selected from the plant genera consisting of Marigold, Tagetes erecta, Tagetes patula, Acacia, Aconitum, Adonis, Arnica, Aquilegia, Aster, Astragalus, Bignonia, Calendula, Caltha, Campanula, Canna, Centaurea, Cheiranthus, Chrysanthemum, Citrus, Crepis, Crocus, Curcurbita, Cytisus, Delonia, Delphinium, Dianthus, Dimorphotheca, Doronicum, Eschscholtzia, Forsythia, Fremontia, Gazania, Gelsemium, Genista, Gentiana, Geranium, Gerbera, Geum, Grevillea, Helenium, Helianthus, Hepatica, Heracleum, Hibiscus, Heliopsis, Hypericum, Hypochoeris, Impatiens, Iris, Jacaranda, Kerria, Labumum, Lathyrus, Leontodon, Lilium, Linum, Lotus, Lycopersicon, Lysimachia, Maratia, Medicago, Mimulus, Narcissus, Oenothera, Osmanthus, Petunia, Photinia, Physalis, Phyteuma, Potentilla, Pyracantha, Ranunculus, Rhododendron, Rosa, Rudbeckia, Senecio, Silene, Silphium, Sinapsis, Sorbus, Spartium, Tecoma, Torenia, Tragopogon, Trollius, Tropaeolum, Tulipa, Tussilago, Ulex, Viola and Zinnia.

37. An animal or human food comprising the genetically modified organism as claimed in claim 25.

38. A method for producing ketocarotenoid-containing extracts or for producing animal and human food supplements comprising cultivating the genetically modified organism as claimed in claim 25 and recovering ketocarotenoid.

39. A ketolase comprising the amino acid sequence SEQ. ID. NO. SEQ ID NO: 2 or a sequence which is derived from this sequence by substitution, insertion or deletion of amino acids and which has an identity of at least 70% at the amino acid level with the sequence SEQ ID NO: 2, with the proviso that the amino acid sequence SEQ ID NO: 2 is not present.

40. A ketolase comprising the amino acid sequence SEQ ID NO: 4 or a sequence which is derived from this sequence by substitution, insertion or deletion of amino acids and which has an identity of at least 70% at the amino acid level with the sequence SEQ ID NO: 4.

41. A nucleic acid encoding a protein as claimed in claim 39, with the proviso that the sequences SEQ ID NO: 1 and SEQ ID NO: 3 are not present.

42. (canceled)

43. A ketolase comprising the amino acid sequence SEQ ID NO: 4 or a sequence which is derived from this sequence by substitution, insertion or deletion of amino acids and which has an identity of at least 65% at the amino acid level with the sequence SEQ ID NO: 4.
